SEQUENCE LISTING (30 sequences)

SEQ ID NO: 1 - protein, 13 aa, Homo sapiens
CASSAGLLGEQYF

SEQ ID NO: 2 - DNA, 39 bp, Homo sapiens
tgtgccagcagtgcggggcttttgggggagcagtacttc

SEQ ID NO: 3 - protein, 13 aa, Homo sapiens
CAIQMYPAYEQYF

SEQ ID NO: 4 - DNA, 39 bp, Homo sapiens
tgtgccattcagatgtacccagcctacgagcagtacttc

SEQ ID NO: 5 - protein, 16 aa, Homo sapiens
CASSYSRGQGSYEQFF

SEQ ID NO: 6 - DNA, 48 bp, Homo sapiens
tgtgccagcagttactcaaggggacaggggtcctatgagcagttcttc

SEQ ID NO: 7 - protein, 13 aa, Homo sapiens
CASRPGQKSMSSS

SEQ ID NO: 8 - DNA, 41 bp, Homo sapiens
tgtgccagcagaccgggacagaaatcaatgagcagttcttc

SEQ ID NO: 9 - protein, 18 aa, Homo sapiens
CASSYSGVLATVDTGELF

SEQ ID NO: 10 - DNA, 55 bp, Homo sapiens
tgtgccagcagttactcgggggtccttgcgaccgtggacaccggggagctgtttt

SEQ ID NO: 11 - protein, 14 aa, Homo sapiens
CASSKARTNKQAVL

SEQ ID NO: 12 - DNA, 46 bp, Homo sapiens
tgtgccagcagcaaagcaaggaccaataaacaatgagcagttcttc

SEQ ID NO: 13 - protein, 13 aa, Homo sapiens
CSARALLSTEAFF

SEQ ID NO: 14 - DNA, 39 bp, Homo sapiens
tgcagtgctagagctttattgagcactgaagctttcttt

SEQ ID NO: 15 - protein, 15 aa, Homo sapiens
CASSLDSSSYNEQFF

SEQ ID NO: 16 - DNA, 45 bp, Homo sapiens
tgtgctagcagcttggactcgagttcttacaatgagcagttcttc

SEQ ID NO: 17 - protein, 14 aa, Homo sapiens
CSVEGPTLYNEQFF

SEQ ID NO: 18 - DNA, 42 bp, Homo sapiens
tgcagcgttgaagggccaacgttgtacaatgagcagttcttc

SEQ ID NO: 19 - protein, 15 aa, Homo sapiens
CASSRPGAGTDTQYF

SEQ ID NO: 20 - protein, 45 aa, Homo sapiens
TGTGCCAGCAGCCGACCGGGAGCGGGCACAGATACGCAGTATTTT

SEQ ID NO: 21 - protein, 16 aa, Homo sapiens
CASRSLTGGGAETQYF

SEQ ID NO: 22 - DNA, 48 bp, Homo sapiens
tgtgccagcaggagcctcactggtgggggggcggagacccagtacttc

SEQ ID NO: 23 - protein, 13 aa, Homo sapiens
CSASASYNSPLHF

SEQ ID NO: 24 - DNA, 39 bp, Homo sapiens
tgcagtgctagcgccagttataattcacccctccacttt

SEQ ID NO: 25 - protein, 13 aa, Homo sapiens
CSAREGGTPGSCF

SEQ ID NO: 26 - DNA, 41 bp, Homo sapiens
tgcagtgctagagaggggggaacaccggggagctgtttttt

SEQ ID NO: 27 - protein, 15 aa, Homo sapiens
CSVEEWASRYNEQFF

SEQ ID NO: 28 - DNA, 45 bp, Homo sapiens
tgcagcgttgaagagtgggctagcagatacaatgagcagttcttc

SEQ ID NO: 29 - protein, 14 aa, Homo sapiens
CASTVDSLDTEAFF

SEQ ID NO: 30 - DNA, 42 bp, Homo sapiens
tgtgccagcaccgtggacagtctggacactgaagctttcttt

(19) United States
(12) Patent Application Publication
(10) Pub. No.: US 2014/0065098 A1
(43) Pub. Date: Mar. 6, 2014

(54) METHOD TO AUGMENT IMMUNE SYSTEM IN RESPONSE TO DISEASE OR INJURY

(71) Applicants: ISRAEL BARKEN, San Diego, CA (US); Derren Barken, San Diego, CA (US)

(72) Inventors: ISRAEL BARKEN, San Diego, CA (US); Derren Barken, San Diego, CA (US)

(21) Appl. No.: 13/971,847

(22) Filed: Aug. 21, 2013

Related U.S. Application Data
(63) Continuation-in-part of application No. PCT/US12/52944, filed on Aug. 29, 2012.

(51) Int. Cl. G01N 33/50; A61K 39/395

(52) U.S. Cl. CPC G01N 33/5008; A61K 39/3955
USPC 424/85.2; 435/6.12

(57) ABSTRACT

In one example, the present invention comprises a deliberate insult or the recognition that an insult has occurred in a patient, which insult induces an immune recognition event. The T cell repertoire is catalogued from samples of the patient's lymphocytes from before and after the insult in order to detect and sequence the TCR alpha and beta loci of highly expanded T cell clonotypes. In some examples, this information is used in turn to generate autologous T cells or to create autologous genetically-engineered T cells, either of which include TCR sequences that target the individual's disease or injury, resulting in cure or amelioration of symptoms.

### CROSS-REFERENCE TO RELATED APPLICATIONS

This application claims priority to U.S. Provisional Application Ser. No. 61/528,657, filed Aug. 29, 2011, and is a continuation-in-part application of International Application PCT/US2012/052944, filed Aug. 29, 2012; both of which are incorporated in their respective entireties herein.

### FIELD OF THE INVENTION

The field of the present invention relates generally to treatments of disease in humans and animals. In particular, the present invention relates to the identification and use of immunostimulatory reagents in such treatments.

### BACKGROUND OF THE INVENTION

Diseases or injury in humans and animals can start by an infectious agent, exposure to noxious chemicals or injurious radiation, exposure to injurious levels of heat or cold, receipt of physical trauma, or by the ordinary workings and errors of an individual's own cells, tissues, organs, and the like. These various sources of disease or injury result in at least one common effect: The human or animal mounts an immunological reaction where the repertoire of T cells of the diseased or injured individual's lymphocyte population may change upon an event that results in matter associated with the disease or injury to rise in concentration in the individual's blood stream or tissue. In consequence of that rise in concentration, the individual's immune system recognizes the presence of a “foreign body” that carries an antigen, which antigen is perceived as new or foreign by the individual's immune system even though the new antigen was made by the individual's own cells. That recognition is referred to as an “immune recognition event” in the description of the present invention herein below.

The immune system's reaction is to attack the bearer of the new antigen, which process includes an upsurge of a particular subset of T cells that share a common receptor. The subset of T cells having the common receptor can be identified by being among the most abundant lymphocytes relative to the total population of lymphocytes in the individual's blood, and being a newly abundant such subset of T cells subsequent to the immune recognition event. The newly abundant subset of such T cells is referred to as a “clonotype.”

Each T cell clonotype mounts an attack on a particular antigen that is perceived as foreign; the act of perceiving an antigen as foreign is what is referred to herein as an “immune recognition event” that triggers downstream effects. Unfortunately, many such immune attacks remain subclinical, providing limited or no good effect to the individual. Nonetheless, the fact that an immune attack began by the individual's built-in system of protection is a signal of a path for ameliorating the particular disease or injury that is harming the individual. Accordingly, if the immune attack mounted by the immune system of an individual suffering from disease or injury is detected in a timely fashion, then a physician could be in a position to administer general and known immune-boosting agents at a time likely to have a good effect. Moreover, if the immune attack is analyzed to identify the path the individual's own immune system is taking, a physician could be in a position to order immuno-reagents specific for the particular patient and his, her, or its particular disease or injury.

The key is building methodologies for identifying when an individual suffering from disease or injury experiences an immune recognition event so that a general and/or personalized immune-boosting treatment can be timely administered for optimum effect. Further methodologies are needed for understanding greater detail of the immune recognition event so that reagents defined by that patient's own immune system can be identified, generated and administered.

Unfortunately, such methods do not exist today. With such methods, one could tackle a wide range of diseases including various cancers, heart disease, and more.

Cancer is a large, heterogeneous class of diseases in which a group of cells display uncontrolled growth, invasion that intrudes upon and destroys adjacent tissues, and often metastasizes, wherein the tumor cells spread to other locations in the body via the lymphatic system or through the bloodstream. These three malignant properties of cancer differentiate malignant tumors from benign tumors, which do not grow uncontrollably, directly invade locally, or metastasize to regional lymph nodes or distant body sites like brain, bone, liver, or other organs.

According to a seminal paper, Hanahan and Weinberg, CELL 100 (1): 57-70 (2000), all cancers share significant characteristics, notably including the ability to evade immune destruction. The field of cancer immunology, which studies this ignoble character of cancer, encompasses the topics of immunosurveillance, immunoediting, immune tolerance, immune escape, and immunotherapy.

Therefore, there is a need for improved methods for treating disease and injury. Learning and applying approaches built into humans and animals that may not reach a clinically detected result is a path of treatment that is currently unavailable. The present invention presents novel methods of analysis and treatment that provides protocols for timely increasing the general attacking power of an individual's immune system and for building efficacy into a quantity of an individual's native lymphocytes that are geared to attack the perceived foreign object as understood by the patient's own immune system.

### SUMMARY OF THE INVENTION

One embodiment provides a method for identifying DNA or RNA sequences of lymphocyte receptors that are present in greater numbers of lymphocytes and/or are more highly expressed after a medical procedure, the method comprising drawing pre-procedure blood from a cancer patient, carrying out a medical procedure on the patient, drawing blood from the patient at one or more times following the medical procedure, purifying lymphocytes from any one or more of the pre-procedure blood draw(s), and/or the post-procedure blood draw(s); isolating DNA or mRNA or both, in the case of the DNA amplifying (if necessary) and sequencing to provide the cell copy number of the TCR gene and in the case of the mRNA assaying for quantity of TCR-encoding messenger to provide the level of expression; dividing RNA units of expression by DNA copy provides level of expression per cell that can be used in treatment. The most direct result of this effort is to identify lymphocytes and/or lymphocyte receptor sequences that have expanded following the medical procedure.

Another embodiment provides a method wherein the patient is selected based on the severity or extent of cancer (Gleason Score for example) and/or patient treatment status.

Another embodiment provides a method wherein the procedure comprises one or more surgical procedure, nonsurgical procedure, or exposure to a drug.

Another embodiment provides a method wherein the cancer is prostate cancer and the medical procedure comprises one or more of cryosurgery, radical prostatectomy, prostate biopsy, radiation therapy, brachytherapy, robotic-mediated radiotherapeutic procedures, electroporation, high frequency ultrasound (HIFU), photodynamic therapy, prostate laser surgery, androgen deprivation therapy, and chemotherapy.

Another embodiment provides a method wherein the procedure leads to an immunogenic response.

Another embodiment provides a method wherein the procedure results in a change in the population lymphocytes.

Another embodiment provides a method wherein the lymphocytes include Tcells displaying various TCRs.

Another embodiment provides a method wherein treatment of cancer comprises inducing in a patient an immunologic response incorporating clonotypes identified by the method of claim 6.

Another embodiment provides a method wherein the method further comprises selecting clonotypes as highly expanded if their frequency (in the measured repertoire) is 0.5% or greater.

Another embodiment provides a method wherein the method further comprises selecting a clonotype that is absent or not highly expanded prior to cryosurgery, but which is highly expanded after cryosurgery as a tumor associated clonotype.

Another embodiment provides a method wherein the method further comprises selecting a clonotype as a tumor specific clonotype if it is highly expanded both before and after a medical procedure, but has a frequency that increases from before to after the procedure, wherein the increase is statistically significant using an appropriate multiple hypothesis testing statistical method to stringently limit the false discovery rate.

Another embodiment provides a method wherein the method further comprises extracting tissue from the patient for use in an in vitro assay of autologous engineered T cells.

Another embodiment provides a method wherein the procedure comprises receptor chain pairing.

Another embodiment provides a method wherein chain pairing is carried out in silico by computer methods.

Another embodiment provides a method wherein chain pairing involves immunology gene alignment software.

Another embodiment provides a method wherein the software is selected from IMGT, JOINSOLVER, VDJSolver, SoDA, iHMMune-align, or other similar tools.

Another embodiment provides a method wherein chain pairing involves using VDJ antibodies.

Another embodiment provides a method wherein the method further comprises obtaining antibodies for the identified segments and use the antibodies to purify a subset of cells which express that gene segment in their (surface) receptors (e.g., using fluorescence-activated cell sorting (FACS) or immunomagnetic selection with microbeads).

Another embodiment provides a method wherein the method further comprises sequencing a subset of cells which have been purified for the desired gene segments.

Another embodiment provides a method wherein chain pairing is carried out using multiwell sequencing or single cell sequencing.

Another embodiment provides a method wherein the method further comprises genetic engineering of autologous T cells, acquired by leukapheresis, to display the TCR or CAR of the induced clonotype(s).

Another embodiment provides a method wherein a T cell is engineered to display a functional TCR.

Another embodiment provides a method wherein a chimeric cell is engineered in which a T cell displays an alternative type of receptor such as a chimeric antigen receptor.

Another embodiment provides a method wherein the method further comprises an in vitro assay.

Another embodiment provides a method wherein the engineered T cells are incubated with tumor tissue or lysate.

Another embodiment provides a method wherein one or more effects are measured during the incubation such as cytokine concentration, cell proliferation, and the like. In some embodiments, the effects of various adjuvants are quantified.

Another embodiment provides a method wherein the engineered T cells are provided as a treatment for cancer.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a flowchart corresponding to protocol for the study of in vitro and in vivo efficacy of autologous engineered T cells.

FIG. 2 shows excerpts from a sample TCR profile report.

FIG. 3 shows excerpts from a sample TCR profile report.

FIG. 4 shows a scatterplot demonstrating the reproducibility of the method, provided by a TCR and BCR profiling vendor (Adaptive Biotechnologies Corporation, Seattle, Wash.).

### DETAILED DESCRIPTION OF THE INVENTION

The present invention is a method of treatment that harnesses a patient's own immune system to effect cure or amelioration of symptoms of disease or injury. The patient that may be successfully treated is human or animal. Diseases and conditions that can be addressed runs the gamut from a cancer to heart disease to an inflammation: Literally, to any disorder, condition, or injury in response to which the body's immune system ordinarily mounts an attack. The immune system is the body's police, truly seeking to bring inner peace by seeking and destroying carriers of foreign antigens. The inventive methods set forth herein and in a sister application collect information from the police (i.e., the body's immune system as observed in the lymphocytes by one skilled in the art and knowledgeable of the present invention) and use that information to (a) time when to administer immunostimulating cytokines in one embodiment, (b) identify lymphocyte clonotypes that can be usefully employed to cure or ameliorate the disease or injury that gave rise to the identified clonotypes, and (c) generate autologous lymphocytes of the identified clonotypes for infusion into the patient for waging an immunotherapeutic attack to a clinically observable level.

The description and examples presented herein below set forth methods and materials learned and designed based on close observation of the body's police force, the immune system. Using the descriptive capabilities of contemporary molecular biology, we have made use of the phenomenal capability to sequence chromosomal DNA from lymphocytes, irrespective whether first purified or not. In particular, the present invention in one embodiment utilizes our ability to focus on the T cell receptor (TCR) gene, especially its V and J regions that are known to those skilled in the art to recombine in a myriad variations and, after recombining those regions, reside respectively within the genome of clonally related lymphocytes, hence that term “clonotypes” already used above. Using well-described primers that target the V and J regions, we can literally sequence those regions and so determine what specific TCRs are present in the population of lymphocytes. Furthermore, the sequence data also provides quantitative data for assessing the relative abundance of lymphocytes in a blood sample having each identified TCR, which in turn tells us the most abundant clonotypes in the blood sample. Observing changes in the most abundant clonotypes in blood samples from before an event that induces an immune recognition event and after that event is used in one embodiment of the present invention. This observation of the body's police tells us which clonotypes are being assembled to handle the insult that proximately induced an immune recognition event (which is the event that results in creating new clonotype lymphocytes geared for addressing the insult).

As will be further described below, there are many sorts of insults. For example, a malignant tumor is itself commonly a silent insult that avoids the body's immune system. But, disruption of the tumor, as occurs in insults to the tumor when conducting a biopsy or chemotherapy or other procedures geared at killing the tumor cells, results in revealing antigens that the immune system sees as foreign, albeit commonly a weak, clinically-limited immune response ensues. But using the methods described here, a physician can assemble an efficacious culture of lymphocytes that have the TCR of the abundant clonotype elicited by the insult visited upon the tumor cells, which resulted in the immune recognition event and the generation of a clonotype that the inventive method showed was being martialed to address the tumor.

Similarly, heightening the efficacy of the immune response that the body mounts in response to a myocardial infarct (physician's name for a heart attack), but which typically results in subclinical effect, i.e., barely or not observed in the ordinary case, will allow the body's own defenses to come to the fore and ameliorate the inflammatory response that exacerbates the oxygen-depriving ischemic event associated with the infarct.

These and other applications of the inventive methods are amply set forth herein. We are not only addressing cancer. As indicated, any disease or injury can be cured or ameliorated as to its symptoms by observing what the policing immune system is trying to do, and then helping the body's internal defenders mount an efficacious treatment along the same lines. While it is clearly contemplated that the present invention is amenable for augmenting the immune system reaction that occurs in the context of any disease or injury, it is worthwhile mentioning some of the diseases that can be addressed. Parkinson's Disease, associated with degeneration of dopamine neurons in the substantia nigra pars compacta part of the brain where the role of inflammation mediates the innate and adaptive immune systems. Panaro and Cianciulli, “Current Opinion and Perspectives on the Role of Immune System in the Pathogenesis of Parkinson's Disease,” Curr. Pharm. Des. 18(2):200-8 (2012). The present inventive method is also amenable for addressing interstitial cystitis and periodontal disease. With regard to periodontal disease, there is little doubt of an immune recognition event occurring with each visit to the dentist's office, particularly if one's teeth are cleaned during the visit. A blood draw before and after such a visit reveals new TCR and BCR abundancies rising in the patient's receptor repertoires, and thus forms the basis for building an immunotherapy to address the inflammation processes acting on that patient's gum tissue. Mentioned elsewhere herein is cancer generally and prostate and breast cancers; also to be highlighted are liver, pancreatic, and kidney cancers, bone cancer, lymphoma, among too many others.

At one level, regarding one embodiment of the present invention, that effort is boosted generally by administering suitable cytokines in suitable combinations at a time point that is proximate to the occurrence of an action that results in an immune recognition event. At a second level, regarding another embodiment of the present invention, that effort is boosted specifically by administering to a patient a heightened concentration of autologous lymphocytes that share the TCR or an optimized engineered form thereof of the more abundant lymphocyte clonotypes that arose in the patient's blood subsequent to the immune recognition event.

In one embodiment, the present invention comprises a medical procedure, and sequencing of the T cell repertoire before and after a medical procedure, in order to detect and sequence the TCR alpha and beta loci of highly expanded T cell clonotypes found in the blood. An important and very helpful variation on selection of source of lymphocytes to be studied in the context of the inventive method is to assess the TCR and BCR repertoire changes before and after an immune recognition event in the bone marrow, which is readily accessed using standard bone marrow aspirations. Obviously, there will be overlap of information derived from the marrow as compared to the cell population circulating in the blood; importantly, the results from a marrow study will also provide information relating to the prognosis of the disease in the patient.

In some embodiments, the differential information relating to the receptor abundancies among lymphocytes is used to create autologous genetically engineered T cells and/or B cells with lymphocyte receptors that target the individual's tumor. Additionally, engineered receptors can also be introduced to the autologous cells and infused into the patient.

Designed immunotherapies have only recently been approved for oncological indications. However, the history of oncology includes interventions which, although designed primarily to destroy cancerous tissue, have in some rare cases had unforeseen, fortuitous, and systemic secondary effects. Specifically, there are rare clinical reports of cryosurgical interventions which, in addition to destroying the targeted cancerous tissue, also resulted in the regression of secondary, metastatic cancerous tissue. It has long been hypothesized that this effect was immunologically mediated. Efforts have been made to enhance its effects using adjuvants, but only limited success has been met.

Separately, the study of the lymphocytic repertoire is being transformed by the advent of high throughput sequencing technologies. Initially, the lymphocyte repertoire was widely studied using techniques such as spectratyping. Recently, and particularly with increased read lengths available, there has been a renaissance in the sequencing of the lymphocyte repertoire. Nonetheless, one embodiment of the present invention purposefully uses spectratyping to assess mixtures of lymphocytes from a patient's blood that include clonotypes that surged in abundance in consequence of the immune recognition event. Such a mixture can be expanded in culture and used to stimulate the patient's immune system without having to sequence any DNA and without having to create an engineered autologous lymphocyte.

Finally, for those embodiments that rely on creating engineered autologous lymphocytes, the tools of genetic engineering/gene therapy have steadily improved. Lymphocytes have been attractive targets for these new techniques, for a variety of reasons. The state of the art in engineering T cells to, for example, carry designed T cell receptors, has advanced dramatically. This has created new opportunities to design the engineered receptors rationally and intelligently.

Described herein are methods for combining a plurality of biological methodologies in new ways to improve the treatment of cancers. In some embodiments, the described methods of treatment incorporate a medical procedure which results in an in situ insult to an individual's tumor (such as cryosurgery). In some embodiments, the methods described herein comprise techniques for analyzing an individual's repertoire of lymphocyte receptors. Also described herein are methods that involve extracting lymphocytes, manipulating them ex vivo for therapeutic purposes, and then returning those lymphocytes to the individual to induce a therapeutic result.

One embodiment of the present invention describes a technique by which a medical procedure (also termed a tumor insult) may be employed to elicit an immunological response. This response may be analyzed in detail by receptor repertoire sequencing of lymphocytes. The analysis may be expected to reveal the receptor sequences of lymphocyte clonotypes which are specific to the cancer. These sequences may be used to genetically engineer lymphocytes which have the same or similar tumor specificity but which may be manipulated ex vivo to enhance their anti-tumor efficacy when returned to the body as an immunotherapy.

In one aspect, the invention combines an in situ insult that induces an immune recognition event, such as freezing, irradiating, or biopsying a tumor, a series of one or more measurements of the T and/or B cell receptor repertoire, an analysis of the T and/or B cell repertoire measurements in order to identify specific T and/or B cell receptor sequences expressed in specific T and/or B cell clonotypes that surged in abundance subsequent to the immune recognition event, and T and/or B cell gene therapy techniques to employ the identified TCR and/or BCR sequences for therapeutic purposes. Autologous T and/or B cells can be engineered using well-established protocols set forth elsewhere herein and well-known to a skilled artisan to express the identified TCR and/or BCR and/or CAR therein; the autologous T and/or B cells can be expanded in culture, again using protocols that are tried and true in the art; and the engineered autologous T and/or B cells once expanded can then be infused or otherwise administered into the patient whose cells they were in the first place.

In another aspect, the invention provides methods for generating an insult to a tumor, thereby inducing an immune recognition event and provoking an immune response which may be measured.

In one aspect, the invention provides methods for measuring said immune response in such a way as to be useful for the design of a gene therapy which is efficacious against a patient's tumor.

In one aspect, the invention provides methods for generating a gene therapy and/or immunotherapy which utilizes the information which is made available by an analysis of sequencing data sets which describe a T cell and/or B cell receptor repertoire.

In one aspect, the invention provides a description of the overall design which combines the individual elements described above into a multi-step clinical strategy which is efficacious.

Aspects of the present invention may be better understood in reference to the Figures.

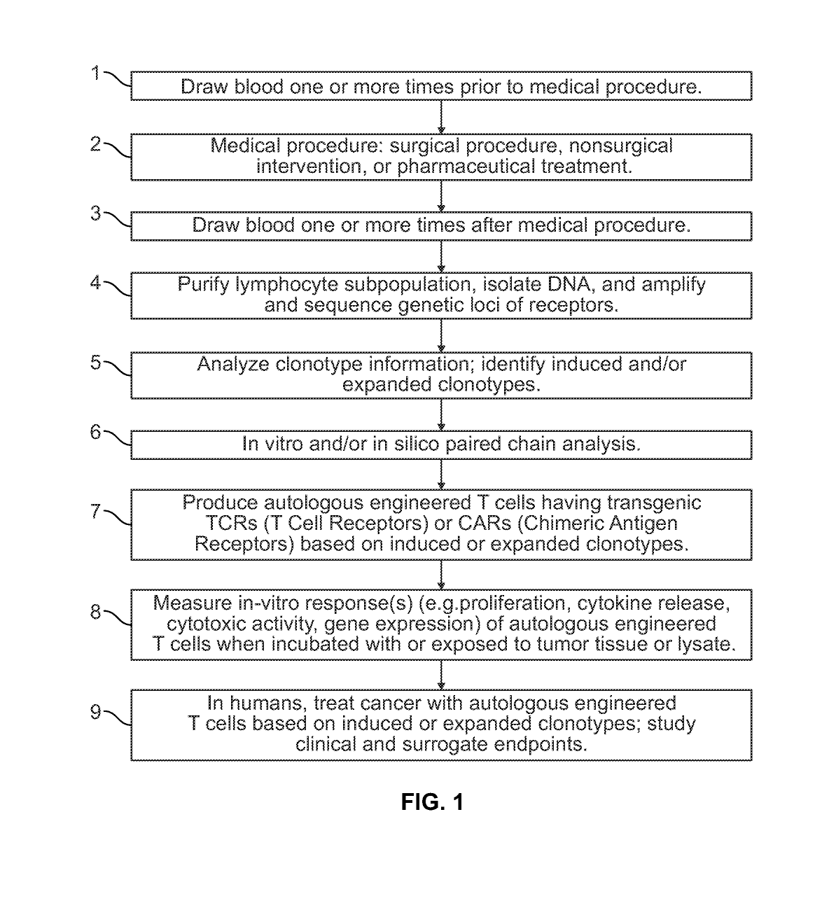

Referring to FIG. 1, this figure describes a method for determining T cell receptors induced or expanded by tumor intervention. However, the figure presents a protocol that can be employed with respect to any disease or injury whose onset or subsequent treatment results in an immune recognition event. Accordingly, although we will present the protocol of the present invention in the context of treatment of a patient afflicted with a tumor, this description is absolutely not intended to be limiting to a tumor treatment only, or to a disease treatment only for that matter, but is usefully considered as description of one embodiment of the inventive method that can be analogously employed with regard to any disease or injury that induces an immune recognition event or that, upon treatment thereof, induces an immune recognition event. In short, the protocol presents a method to identify the response of the patient's own immune system and then augment that line of attack to achieve a clinical effect resulting in cure or amelioration of symptoms.

Further regarding the content of FIG. 1, the recital there of a series of steps relates to just one embodiment of the present invention. It is provided here as a tool for discussing one way to practice the invention. However, it is not the only embodiment of the invention. For example, we have determined that the lymphocyte purification step is optional because tools are available that allows the following steps to proceed without difficulty, as is known in the art. Other steps can vary as well, as one can perceive in viewing the structure of our claims below, where fewer than all of the steps set forth in FIG. 1 are included in our broader claims, which claims as recited describe a completely operative invention. We also want to mention that the order of the steps set forth in FIG. 1 are not written in stone: Logic dictates, of course, that a blood or tissue sample from prior to an immune recognition event necessarily is drawn prior to drawing a blood or tissue sample from after the immune recognition event. But analyzing the included TCRs or BCRs or V/J segments in the earlier taken sample need not be done until it is needed for comparing to the analogous result from the later taken sample.

FIG. 1 describes an in vitro (steps 1-8) and in vivo (step 9) study. Patients may be selected for the study based on the severity or extent of their cancer (Gleason Score for example), their hormonal treatment status (androgen deprivation therapy, for example), and the like. In some embodiments, a population of patients are selected for study that statistically represent the patient population as a whole and/or a subset of the patient population suitable for treatment using the methods described herein. In the first step, one or more pre-operative blood draws are taken from a patient afflicted with cancerous tissue. These blood draws may be analyzed immediately or preserved for later analysis using any suitable method known to the art that preserves the integrity of the contained cells so that a later access of the contained genomic DNA can be subjected to DNA sequencing and analysis.

In a second step, the cancerous tumor and/or cancerous tissue of the patient(s) are “insulted” or “intervened” or “treated,” meaning that the tissue is acted upon, treated, surgically altered, altered by radiation or other nonsurgical intervention, or exposure to a drug and the like. Any medical procedure known in the art of cancer treatment is appropriate, and may include various types of prostate surgery in various embodiments. Non-limiting examples include one or more of cryosurgery, radical prostatectomy, prostate biopsy, radiation therapy, brachytherapy, robotic-mediated radiotherapeutic procedures, electroporation, high frequency ultrasound (HIFU), photodynamic therapy, prostate laser surgery, androgen deprivation therapy, and chemotherapy. In some embodiments, a method of tumor insult is selected that is known to lead to an immunogenic response. Without being limited to any particular theory, it is hypothesized that intervention in the tumor will result in a change in the population lymphocytes. The lymphocytes include T-cells displaying various T cell receptors (TCRs). In some embodiments, TCRs that are specific to the cancerous tumor can be utilized in methods for treatment of the cancer. In some embodiments, the cancerous tissue is analyzed immediately or preserved for later analysis. Methods of analysis can include methods described herein or any suitable method of genetic and/or biochemical analysis known to those skilled in the art.

In other embodiments, the tissues are preserved, optionally as formalin-fixed, paraffin embedded (FFPE) tissue.

In a third step of the method depicted in FIG. 1, blood is drawn from the patient at various times following intervention of the cancerous tissue. Any time period may be suitable and may be adjusted to coincide with the timing of an immunological response in the patient. In some embodiments, blood is drawn at a plurality of times. In some embodiments, blood is drawn on the same day as the intervention, and again at 2 days, 3 days, 5 days, 7 days, 10 days, 14 days, 21 days, 30 days, and the like following surgery. The post-operative and/or post-intervention blood samples may be analyzed immediately or preserved for later analysis.

In Step 4 of FIG. 1, lymphocytes are separated and purified from the drawn blood. One example method of lymphocyte separation comprises layering heparinized venous blood onto a density gradient of Ficoll-Isopaque (GE Healthcare Biosciences, Pittsburgh, Pa.) in 15 ml conical tube in the ratio 3:1; centrifuging the tube at about 1800 rpm for about 20 minutes; removing the middle layer; transferring to another tube; centrifuging the tube at about 2000 rpm for about 5 minutes to about 15 minutes; discarding the supernatant; suspending the pellet in RPMI-1640 medium (Life Technologies Corporation, Grand Island, N.Y.); washing the cells twice with the RPMI-1640 medium; suspending the cells in about 1 ml of the RPMI-1640 medium; dividing the cell suspension into two tubes; incubating the suspensions with fluorescent reagents such as monoclonal antibodies specific for CD4 or CD8 cells, which antibodies are tagged with a fluorochrome, such as phycoerythrin (PE) or 5(6)-fluorescein isothiocyanate (FITC) (BD Biosciences Pharmingen, San Diego, Calif.); vortexing the suspension at room temperature; incubating the suspension for 2 hours in the dark at room temperature; and identifying the specific lymphocytes based on staining with specific antibodies for sorting on a cell sorter apparatus such as the BD FACStar Plus (BD Biosciences, San Jose, Calif.). One protocol for separating and purifying the lymphocytes is set forth in Toor and Vohra, Immune responsiveness during disease progression from acute rheumatic fever to chronic rheumatic heart disease, MICROBES AND INFECTION (2012), in press (available online at http://dx.doi.org/10.1016/j.micinf.2012.07.003).

Separation and purification can also be accomplished by immunomagnetic selection with microbeads. The immunomagnetic selection can be done with commercially available kits such as Miltenyi CD8+ kits (Miltenyi Biotec Inc., Auburn, Calif.). Other separation methods such as fluorescence-activated cell sorting (FACS) can also be used to separate and purify lymphocytes from the drawn blood. Lymphocyte isolation may be done using positive isolation, negative isolation, or a sequence of steps that includes both positive and negative isolation. For example, CD3+ T Cells can be separated using an Invitrogen Dynabeads CD3 kit (Catalog #11151D or 11365D, available from Life Technologies Corporation, Grand Island, N.Y.), or the CD3 MicroBeads or CD56 MultiSort Kit from Miltenyi Biotec Inc. (Auburn, Calif.), or other such kits designed for isolation of other lymphocytes; other suitable separation methods can also be used. For non-magnetic separations, a Pluriselect kit (Catalog #10-00300-21, Pluriselect GmbH, Leipzig, Germany) may be used. Numerous kits from the mentioned manufacturers and other manufacturers may be used for negative selection. In addition, more automated tools such as the Miltenyi Biotec autoMACS Pro Separator (Miltenyi Biotec Inc., Auburn, Calif.) may be used.

In Step 4 of FIG. 1, DNA is isolated from any one or more of the pre-operative blood draw(s), the cancerous tissue, and/or the post-operative blood draw(s). In some embodiments, DNA is extracted from a mixed tissue or mixed cell-type sample, optionally from whole blood or cancerous tissue. This embodiment may eliminate the need for certain sample processing steps, whereby the genetic loci of interest can be interrogated from a mixed DNA sample. In another embodiment, the blood and/or tissue samples are first enriched for certain lymphocytes, optionally by whole blood fractionation. Whether from an enriched sample, or from a non-enriched sample, DNA can be isolated according to any suitable method known to those skilled in the art. Ones skilled in the art will be aware that kits such as QiA DNA Blood Maxi prep #51192 or QIAGEN DNeasy Blood and Tissue kit #69506. CDNA may also be prepared with kits such as QIAGEN FastLane Cell cDNA Kit #215011. The identified kits are available commercially from QIAGEN Inc. (Sunnyvale, Calif.).

In step 4 of FIG. 1, the extracted DNA is then amplified (if necessary) and sequenced. Specifically, the DNA encoding the lymphocyte receptors is amplified in some embodiments, optionally the T cell receptors. T cell receptors consist of alpha (α) and beta (β) chains. In some embodiments, both the alpha and beta chains of a TCR are amplified. To amplify a section of DNA using PCR, one requires two primers. Primers are short strands of DNA that physically stick (or anneal) to the ends of the DNA, and allow other molecules (known as polymerases) to “copy” what is in between them. In other embodiments, various loci can be amplified separately. For example, the alpha and beta chains of a TCR can be amplified separately to yield two PCR products. Skilled persons will be familiar with methods in polymerase chain reaction (“PCR”) that are suitable for DNA amplification including design of short, single-stranded pieces of DNA that serve as PCR primers, adjustment of annealing, melting and extension times and temperatures and the like such that high quality PCR products are produced. Some DNA amplification procedures may be conducted in multi-well plates.

Step 4 of FIG. 1 includes DNA sequencing of the DNA. Methods of DNA sequencing are well known in the art and have improved rapidly in recent years in features such as read length, improved throughput, reduced cost and the like. One suitable method of DNA sequencing is pyrosequencing. Pyrosequencing is a method of DNA sequencing (determining the order of nucleotides in DNA) based on the “sequencing by synthesis” principle. It differs from Sanger sequencing, in that it relies on the detection of pyrophosphate release on nucleotide incorporation, rather than chain termination with dideoxynucleotides as used in the Sanger method. DNA sequencing results in sequence data of adenine (A), cytosine (C), thymine (T) and guanine (G) that is analyzed by computer methods. Sequencing may be done by vendors such as Adaptive Biotechnologies Corporation (Seattle, Wash.).

Step 5 includes methods for identifying lymphocytes and/or lymphocyte receptor sequences that have expanded following intervention of the cancerous tissue. For most locations along the DNA, the sequence is identical from cell to cell. However, the T cell receptor and B cell receptor locations are different. DNA regions coding for T cell receptor and B cell receptor, the body uses a process known as somatic or “VDJ” recombination to recombine the DNA at that location (in one individual cell). Therefore, if one takes a population of T cells or B cells (from a blood draw), and carries out a targeted resequencing of the DNA at the T cell or B cell receptor locus, one will get many different “reads”—or sequences of DNA (each read comes from a different cell). When performing targeted resequencing a mixture of primers are used, which includes a plurality of primers. This provides for “multiplexed” PCR. The primers correspond to the DNA at either end of the DNA targeted for amplification. In this way, the primers act as “bookends.” The bookends vary from cell to cell, but the possible bookends come from a small, fixed set. In the case of T cell and B cell receptors, there are known bookends called V segments and C (constant) segments and because the resequencing is done using a mixture of primers, all the possibilities are covered. Every T cell receptor or B cell receptor will include one V segment and one C segment. The DNA between the bookends is amplified and the amplified DNA may then be sequenced using well known techniques such as pyrosequencing. These sequences represent a “repertoire” which can be analyzed. In particular, one may decipher which sequences are more or less common. In addition, one may take two blood draws (“before” and “after”) and decipher which sequences were absent before a medical procedure but appeared after the medical procedure. By “expansion” it is meant that the number of members of a clonotype is greater relative to other clonotypes found in the same blood sample following intervention or injurious event that induces an immune-recognition event; as compared to the clonotypes identified in a blood sample collected from the same patient before intervention or injurious event. Expansion can be quantified by comparing the amount of amplified DNA for a given alpha and/or beta chain from samples before and after intervention or injurious event.

The methods of Step 5 are often computer-based. In some embodiments, clonotypes are considered highly expanded if their frequency (in the measured repertoire) is 0.5% or greater. In some embodiments, a clonotype which is absent or not highly expanded prior to cryosurgery or other medical procedure, but which is highly expanded after cryosurgery or other medical procedure, is inferred to be a tumor associated clonotype. In some embodiments, a clonotype will be inferred to be a tumor specific clonotype if it is highly expanded both before and after tumor intervention, but has a frequency that increases from before to after cryosurgery or other medical procedure, where the increase is statistically significant using an appropriate multiple hypothesis testing statistical method to stringently limit the false discovery rate.

In other embodiments, sequencing is replaced with spetratyping (also known as immunoscoping). Spectratype analysis takes advantage of PCR technology to amplify template cDNA corresponding to rearranged transcripts with different complementarity determining region 3 (CDR3) lengths from specific TCR variable region genes in a competitive manner. Persons of ordinary skill in the art are familiar with protocols for spectratype analysis, such as those available at http://www.currentprotocols.com/WileyCDA/CPUnit/refid-1028.html. A protocol for spectratype analysis employed in the context of the present invention is presented herein in Example 4.

When treating a patient by the instant inventive method using spectratype analysis, the spectratype analysis of a patient's lymphocytes is carried out before and after an immune recognition event, such as a medical procedure intervention or injury event, to identify the particular lymphocyte receptor V and/or J segments that expanded subsequent to the immune recognition event. A subset of cells expressing the identified V and/or J segments can then be isolated from the patient's own blood, expanded, stimulated, or modified ex vivo, and then reinfused as a therapeutic treatment.

It is understood that FIG. 1 is a depiction of generalized procedures and not limiting. For example, Step 4 of FIG. 1 does not limit the method to performing DNA extraction, amplification and sequencing simultaneously. Furthermore, there may be other steps not necessarily depicted in the figures, such as sample processing and the like; and the listing of steps in a given order is not in and of itself a recital that the identified steps must be accomplished in that order, irrespective of how they are listed or described in the detailed description, figures, examples, or, for that matter, claims. For example, one of ordinary skill necessarily understands that steps 4 and 5 comprising the steps of: purification; DNA isolation; amplification; sequencing; and identification of lymphocyte clonotypes may be carried out immediately after each blood draw or blood samples drawn before an immune recognition event can be stored and processed per steps 4 and 5 at the same time that a blood sample is drawn subsequent to the immune recognition event.

In some embodiments, Step 5 of FIG. 1 results in separate data comprising alpha chains that are induced upon intervention of the tumor and beta chains that are induced upon intervention of the tumor.

Step 6 of the procedure depicted in FIG. 1 involves “paired chain analysis”. In this step, various methods can be utilized to pair induced alpha and beta chains such that the pairing results in a TCR that binds to an epitope of the cancerous tissue or otherwise leads to an immune response targeting the cancerous tissue. In some embodiments post-sequencing pairing may be unnecessary or relatively simple, for example in embodiments in which the alpha and beta chain pairing information is not lost in the procedure, such as if one were to sequence from single cells.

In some embodiments, the chain pairing may be assisted in silico by computer methods for annotating VDJ gene segments. For example, specialized, publicly-available immunology-directed gene alignment software is available from International IMmunoGeneTics information system (IMGT; Lefranc, et al., NUCL. ACIDS RES. 37:D1006-D1012 (2009)), including V-Quest (Brochet, et al., NUCL. ACIDS RES. 36:W503-508 (2008)) and JunctionAnalysis (Yousfi Monod, et al., BIOINFORMATICS 20:1379-i385 (2004)); JOINSOLVER (Souto-Carneiro, et al., J. IMMUNOL. 172(11):6790-6802 (2004)); VDJSolver (Ohm-Laursen, et al., IMMUNOLOGY 119(2):265-77 (2006)); Somatic Diversification Analysis (SoDA; Volpe, et al., BIOINFORMATICS 22(4):438-44 (2006)); iHMMune-align (Gaeta, et al., BIOINFORMATICS 23:1580-1587 (2007)), and other similar tools.

In some embodiments, the chain pairing may be done using VDJ antibodies. For example, one may obtain antibodies for the identified segments and use the antibodies to purify a subset of cells which express that gene segment in their (surface) receptors (e.g., using FACS, or immunomagnetic selection with microbeads). One may then sequence from this subset of cells which have been purified for the desired gene segments. If necessary, this secondary sequencing may be done more deeply (i.e. at a higher resolution) than the first round of sequencing. In this second sequence data set, there will be far fewer induced clonotypes, greatly easing the task of chain pairing. Depending on the gene segments, there may be only one induced alpha chain and one induced beta chain for example.

In some embodiments, the chain pairing may be done by trial and error.

In some embodiments, the chain pairing may be done using multiwall sequencing. For example, one may isolate gene segment purified cells or unpurified cells into a microwell plate, where each microwell has a very low number of cells. One can amplify and sequence the cells in each well individually, which provides another means to pair the chains of interest by sequencing on a single cell basis, facilitating the pairing of induced alpha and beta chains.

One skilled in the art necessarily understands that purification of lymphocytes may be performed after each blood draw or may be performed after the last blood draw has been completed. Moreover, identification of lymphocyte clonotypes may also follow after each blood draw or after the completion of the intended blood draws with respect to the analysis surrounding the immune recognition event.

Step 7 of FIG. 1 includes genetic engineering of autologous T-cells to display the TCR or chimeric antibody receptor corresponding to the induced clonotype(s). Methods of genetic engineering are generally known in the art and can be found in well-known texts of protocols including Sambrook et al. (2001), MOLECULAR CLONING. A LABORATORY MANUAL, Cold Spring Harbor Press, Plainview, N.Y.), to provide one non-limiting example. The alpha and beta chains of the T-cells of this invention may be expressed independently in different hosts or in the same host. Preferably the alpha and beta chains are introduced into the same host to allow for formation of a functional T-cell receptor in the host cell. In some embodiments, the host cell is capable of inducing an immune response in a patient. The means by which the vector carrying the gene may be introduced into the cell include, but are not limited to, microinjection, electroporation, transduction, retroviral transduction or transfection using DEAE-dextran, lipofection, calcium phosphate, particle bombardment mediated gene transfer or direct injection of nucleic acid sequences encoding the T cell receptors of this invention or other procedures known to one skilled in the art and set forth in well-known and utilized sourcebooks such as Sambrook et al., Id.

Genetic engineering of human T cells may also be accomplished using lentiviral vector gene transfer, as is well-described in the art. Protocols for this method are known to one skilled in the art. See, e.g., Verhoeyen et al., “Lentiviral Vector Gene Transfer into Human T Cells,” METHODS MOL BIOL. 506: 97-114 (2009).

In some embodiments, a T-cell is engineered to display a functional TCR. In other embodiments, a chimeric cell may be engineered in which a T-cell displays an alternative type of receptor such as a B-cell receptor.

Step 8 of FIG. 1 includes in vitro assays. In some embodiments, the autologous engineered T-cells from Step 7 of FIG. 1 are incubated with tumor tissue or lysate. In various embodiments, various effects are measured during the incubation such as cytokine concentration, cell proliferation, and the like. In some embodiments, the effects of various adjuvants are quantified.

Turning now to step 9 of FIG. 1, depicted herein is an exemplary sequence of steps which comprise a therapeutic strategy. T cell receptors may be applied in a treatment for cancer using T cell autologous or allogenic adoptive transfer cell therapy. In this method, T cells from the patient (autologous) or a donor (allogeneic) are “retargeted by a process of engineering, in which the endogenous T cell receptor is suppressed, and an alternative T cell receptor sequence (as determined previously) is introduced into the cell. These retargeted T cells are reinfused into the patient and monitored for efficacy in destroying the targeted cells.

In some embodiments, the cells further include one or more adjuvants suitable to illicit or amplify an immune response.

In step 9, the effects of the treatment are evaluated by reference to clinical and surrogate endpoints. The clinical endpoints may include one or more of overall survival, progression free survival, or tumor regression. The surrogate endpoints may include longitudinal measurements of cancer biomarkers. In the case of prostate cancer, available surrogate endpoints would include PSA (prostate specific antigen) and circulating tumor cells.

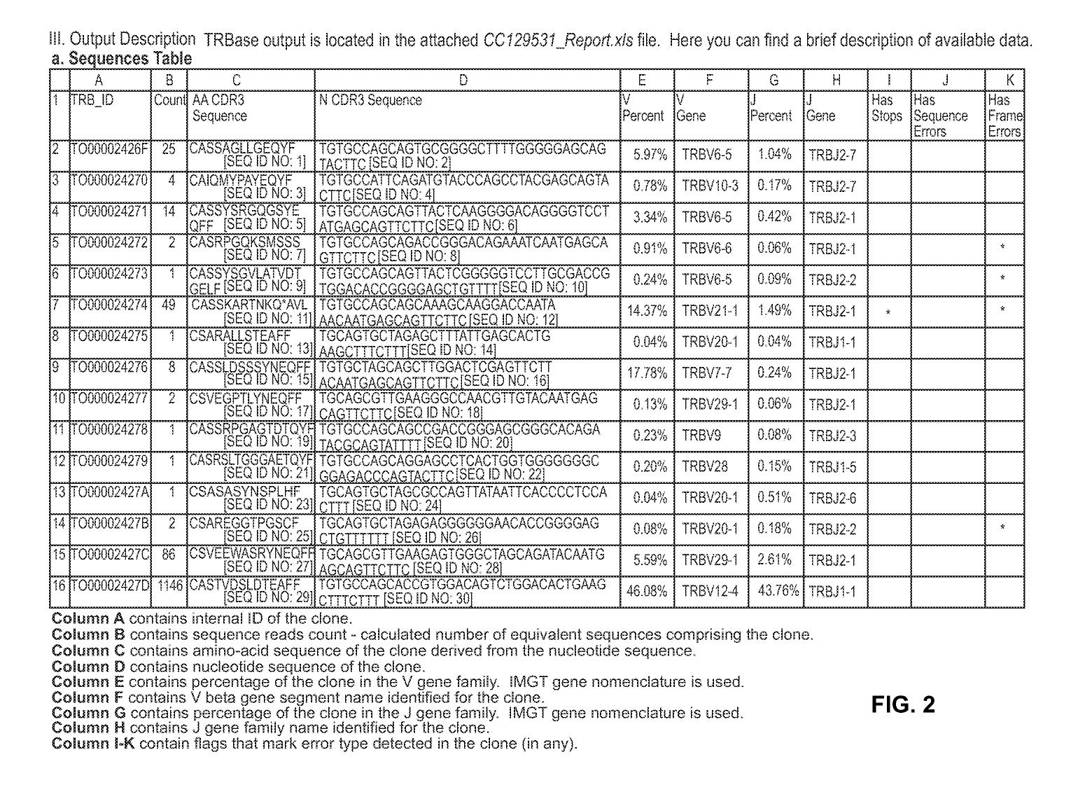

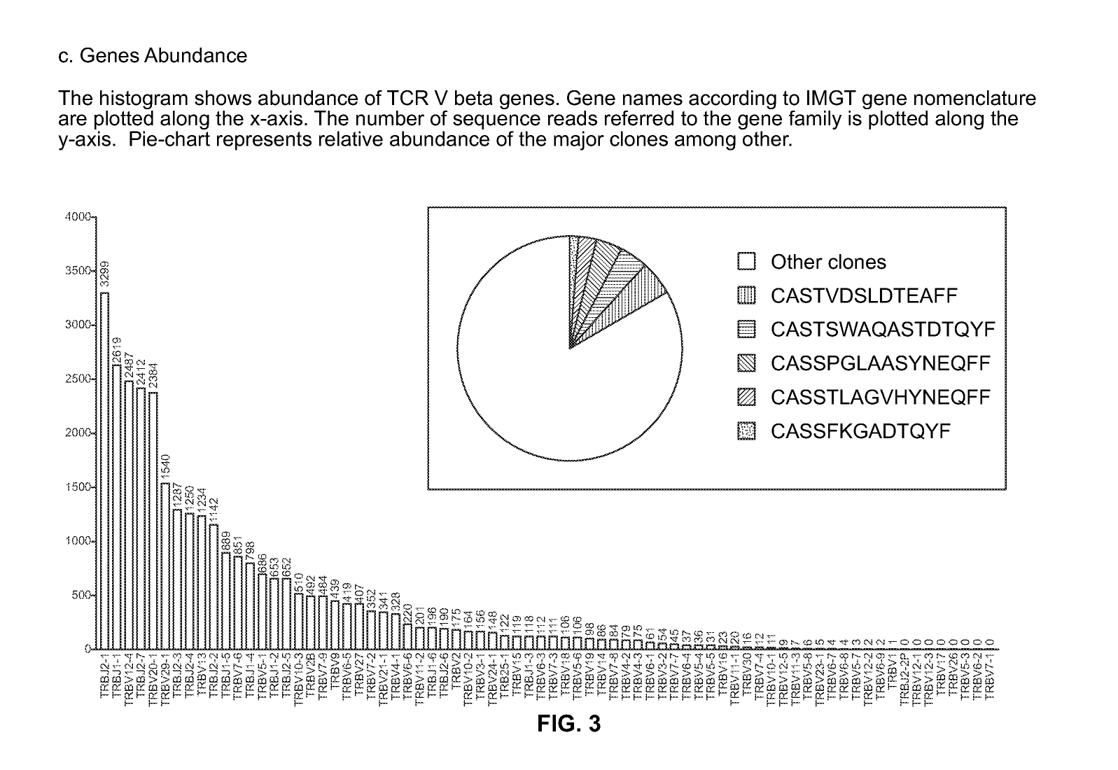

FIGS. 2 and 3 show that the TCR beta chain complementarity determining region 3 (CDR3) sequences and clonotype frequencies are commercially available. In these examples, the commercial provider is Evrogen Lab (Moscow, Russia).

FIG. 2 is an exemplary report showing among other things, a listing of clonotypes (clones), their sequence and read count, the percentage of the clone in the V gene family, the percentage of the clonotypes in the J gene family.

FIG. 3 the abundance of T-cell receptor V beta genes depicted as a histogram and a pie chart. FIGS. 2 and 3 are examples of computer-based methods that can be used to identify T-cell receptor segments that increase in abundance following insulting the tumor.

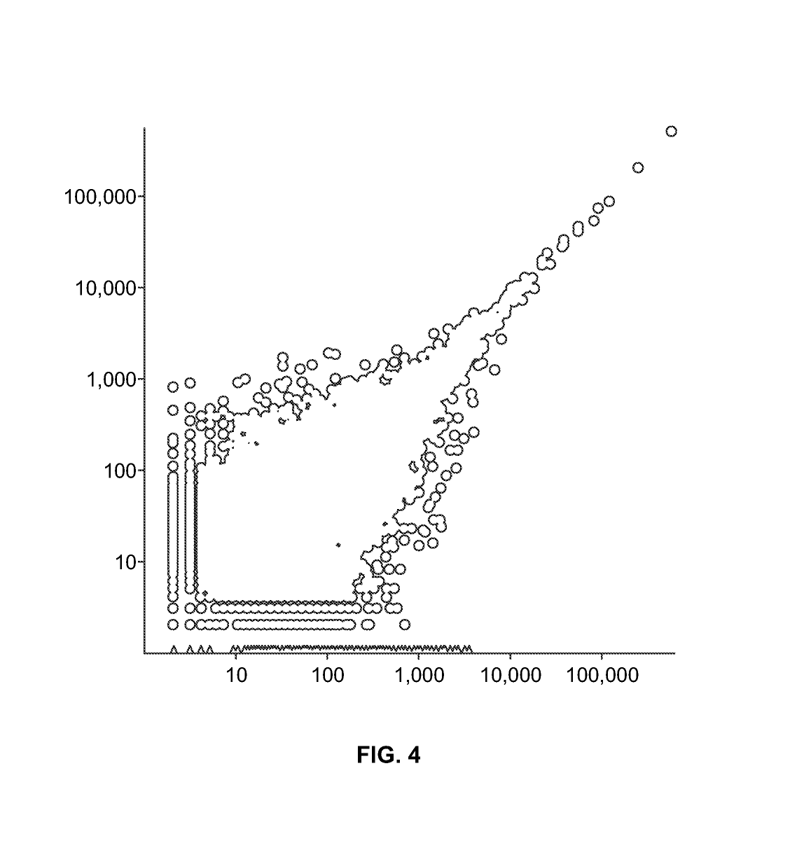

FIG. 4 shows a scatterplot demonstrating the reproducibility of the method. In this case, both the x-axis and y-axis are the same logarithmic scale plotting the same data, so perfectly reproducible data would fall on a 45 degree line ascending from the bottom left to the top right of the graph. FIG. 4 shows that a majority of the data points follow this 45 degree trend of reproducibility.

The methods disclosed herein can be used to treat all types of cancer including but not limited to breast cancer, colon cancer, liver cancer and the like.

Either the primary or secondary tumors can be insulted.

In some embodiments, lymph material is removed or drawn from the patient in lieu of blood.

One embodiment provides a method for identifying the DNA or RNA sequences of lymphocyte receptors expressed by lymphocytes that are present in increased numbers in a particular patient after a medical procedure; the method comprising: (i) drawing blood at one or more times from a cancer patient prior to a medical procedure (ii) carrying out a medical procedure; (iii) drawing blood from the patient at one or more times following the medical procedure; (iv) purifying a lymphocyte subpopulation, isolating DNA, amplifying (if necessary) and sequencing the genetic loci of receptors; and (v) identifying lymphocytes and/or lymphocyte receptor sequences that have expanded in number following the medical procedure.

Another embodiment provides a method wherein the analysis of lymphocyte receptor repertoire sequences, from patient samples obtained both before and after a medical procedure, enables the production of autologous genetically engineered T cells having transgenic T cell receptors which are specific to the patient's cancer.

Another embodiment provides a method wherein the analysis of lymphocyte receptor repertoire sequences, from patient samples obtained both before and after a medical procedure, enables the production of autologous genetically engineered T cells having chimeric antigen receptors which are specific to the patient's cancer.

Another embodiment provides a method wherein autologous engineered T cells, having transgenic T cell receptors (TCRs) or chimeric antigen receptors (CARs), are shown to be efficacious in vitro in mounting a cytotoxic and/or therapeutic immune response to the presence of tumor tissue or lysate.

Another embodiment provides a method wherein autologous engineered T cells, having transgenic T cell receptors (TCRs) or chimeric antigen receptors (CARs), are shown to be efficacious in vivo in affecting clinical endpoints such as overall survival, progression free survival, or tumor regression.

Another embodiment provides a method wherein autologous engineered T cells, having transgenic T cell receptors (TCRs) or chimeric antigen receptors (CARs), are shown to be efficacious in vivo in affecting surrogate endpoints such as longitudinal measurements of biomarker levels or circulating tumor cells.

Another embodiment provides a method wherein the patient is selected based on the severity or extent of cancer (for example, a Gleason score in the context of prostate cancer), patient treatment status (for example, androgen deprivation therapy status in the context of prostate cancer), or other clinical status.

Another embodiment provides a method wherein the medical procedure, comprises one or more surgical procedures, nonsurgical interventions or pharmaceutical treatments.

Another embodiment provides a method wherein the cancer is prostate cancer and the medical procedure comprises one or more of cryosurgery, radical prostatectomy, prostate biopsy, radiation therapy, brachytherapy, robotic-mediated radiotherapeutic procedures, electroporation, high frequency ultrasound (HIFU), photodynamic therapy, prostate laser surgery, androgen deprivation therapy, and chemotherapy.

Another embodiment provides a method wherein the intervention leads to an immunogenic response.

Another embodiment of the present invention includes an onset of disease or injury or an intervention/treatment thereof, where a change in the population or repertoire of lymphocytes is observed and used to generate immunostimulating reagents. The immunostimulating reagent, in one such embodiment, can be one or more cytokines administered proximately to the onset of disease or injury for a general immunostimulating effect at a time when the immune system is usefully stimulated to react to the recently onset disease or injury. In a second embodiment, the immunostimulating reagent can be lymphocytes having a TCR or CAR consistent with one of the identified newly abundant clonotypes identified in accordance with the present invention.

Another embodiment provides a method wherein the lymphocytes include B cells displaying various B Cell Receptors (BCRs).

Another embodiment provides a method further comprising selecting clonotypes as highly expanded or newly abundant if their frequency (in the measured repertoire) is 0.5% or greater.

Another embodiment provides a method further comprising selecting a clonotype that is absent or not highly expanded prior to a medical procedure, but which is highly expanded or abundant relative to other clonotypes in the patient's blood sample drawn after a medical procedure, which selected clonotype is referred to as a treatment-associated clonotype.

Another embodiment provides a method further comprising selecting a clonotype as a tumor specific clonotype if it is highly expanded both before and after a medical procedure, but has a frequency that increases from before to after a medical procedure that disrupts the patient's tumor, wherein the increase is statistically significant using an appropriate multiple hypothesis testing statistical method to stringently limit the false discovery rate.

Another embodiment provides a method wherein the sequences of two chains comprising a lymphocyte receptor (e.g., alpha and beta TCR, gamma and delta TCR, or heavy chain and light chain BCR) are paired. The chain pairing is carried out in silico by computer methods, or by use of immunology gene alignment software, or by using VDJ antibodies in accordance with knowledge generally known in the art, or by using multiwell sequencing as known in the art, or by manual means for assessing the DNA sequences of expected chain pairs as know in the art; wherein, in one embodiment, the software is selected from IMGT, JOINSOLVER, VDJSolver, SoDA, iHMMune-align, or other similar software tools.

Another embodiment provides a method comprising obtaining antibodies for the identified VDJ gene segments and using the antibodies to purify a subset of cells which express that gene segment in their (surface) receptors (e.g., using FACS, or immunomagnetic selection with microbeads).

Another embodiment provides a method further comprising sequencing a subset of cells which have been purified for the desired VDJ gene segments.

Another embodiment provides a method further comprising genetic engineering of autologous T-cells to display the TCR of the induced clonotype(s), or to display a functional TCR, or to display an alternative type of receptor, such as a B-cell receptor. In one further embodiment, the engineered autologous T cells are incubated with tumor tissue and/or lysate of tumor tissue prior to administering same to the patient by infusion or other in vivo treatment. One or more criteria are commonly measured during the incubation, such as cytokine concentration, cell proliferation, and the like. In some embodiments, the effects of various adjuvants are quantified. After administration of the engineered autologous T cells, overall survival, progression free survival, tumor regression, and other clinical end points are measured and recorded. In another embodiment, the clinical benefit of treatment with engineered autologous T cells is measured in terms of surrogate endpoints such as longitudinal measurements of biomarker levels or circulating tumor cells.

In one embodiment, lymphocytes bearing particular V and/or J segments are filtered and isolated in a filtration and purification step and incubated for autologic or allogenic cell transfer therapy without genetically engineering lymphocytes to express specific receptors on the cell surface. For this embodiment, spectratyping is carried out on one or more of T cell receptor alpha chains, beta chains, gamma chains, or delta chains; or, in the same fashion, B cell chains can be the subject of the spectratyping. As a result of spectratyping, a change in amount or composition (ologiclonality) of one or more V or J segments in one or more chains is identified if it exists in the differences between the before and after immune recognition event blood samples.

For example, following a cryosurgery, which is a medical procedure that induces an immune recognition event, a qualitative or quantitative increase in amount and oligoclonality may be seen in segments TRAV8-2, TRAJ4, TRBV17, and/or TRBJ2-5 (as defined in the book The T Cell Receptor FactsBook, by Marie-Paule Lefranc and Gerard Lefranc). Consequently, using appropriate commercial antibodies which are specific to the V and/or J segments identified, cells bearing receptors containing these segments are isolated by a standard and known method for sorting cells, such as fluorescence activated cell sorting (FACS), magnetic beads (Life Technologies dynabeads), or related methods (pluriSelect GmbH cell isolation technologies, to name another).

These autologous cells, bearing desired V and/or J segments, are a “subpopulation” or “subcompartment” of the overall T cell or B cell repertoire. The isolated subcompartment or subpopulation of T cells may then be manipulated or activated in vivo using a number of methods which will be known to one skilled in the art, including cytokines, anti-CD3/anti-CD28 antibodies (Catalog ##111-31D, 111-32D, 111-61D; Life Technologies Corporation, Grand Island, N.Y.), etc. These methods are used, for example, in T cell proliferation assays.

Finally, the relevant subcompartment or subpopulation of cells, possibly having been expanded, activated, or manipulated in vitro, may be therapeutically reinfused. Their persistence and status may also be monitored post-infusion, with subsequent blood draws, as will be known to one skilled in the art. It is notable that this particular embodiment is relatively rapid and cost-effective, since spectratyping may be used instead of sequencing, and isolation using V and J specific antibodies may be used instead of genetic engineering. Further, by avoiding genetic engineering, this embodiment avoids problems with unwanted chain pairings.

In another embodiment, both spectratyping and sequencing may be done, where spectratyping reveals the V and J segments of interest, and sequencing is done using only primers which correspond to the V and J segments of interest. This approach greatly reduces the amount of sequencing necessary, and, as well, provides the opportunity to utilize more rapid sequencing technologies.

In another embodiment a “panning experiment” is carried out after identifying expanded receptors before and after sequencing in which many copies of the identified receptor are incubated with a combinatorial library of possible epitopes. Once the corresponding epitope is identified, tetramers loaded with the identified epitope are used to isolate lymphocytes from the patient's own cells which are specific to that epitope. These cells can then be expanded ex vivo, manipulated, and then therapeutically infused. One skilled in the art is familiar with the procedure (Li Pira et al., “High Throughput T Epitope Mapping and Vaccine Development,” J. BIOMED. BIOTEHNOL. 2010:325720 (2010); Sung et al., “T-cell Epitope Prediction with Combinatorial Peptide Libraries,” J. COMPUT. BIOL. 9(3):527-39 (2002).

### DEFINITIONS

All technical terms have the standard accepted meaning in the art to which the present disclosure applies. Certain definitions may be found in U.S. Pat. No. 5,830,755, which is incorporated herein with respect to defining technical terms, however in the event that there is any discrepancy between term definitions expressly set forth herein and those in the referenced patent, the definitions expressly set forth herein shall govern.

“Cryotherapy” is the local or general use of low temperatures in medical therapy or the removal of heat from a body part. “Cryoablation” is a process that uses cold (cryo) to destroy tissue (ablation). In cryoablation, the destroyed tissue typically remains in the body, enhancing an immune response.

A “lymphocyte” is a type of white blood cell in the vertebrate immune system that is used herein to refer to both T cell and B cell varieties of lymphocytes, either of which are amenable to the methods of the present invention; moreover, protocols described with reference to either a T cell or a B cell are equally available for application to the other lymphocyte variety. Lastly, as used in the immunological arts, T cells and B cells are called “small” lymphocytes, and “large” lymphocytes include macrophages and granular lymphocytes, such as natural killer cells (NK cells). For the purposes of clarity in the specification of our invention, we refer to the small lymphocytes only when we recite the term “lymphocyte” herein; and when we intend to refer to a large lymphocyte as that term is understand in the art, we will recite the name of the particular cell.

“T lymphocytes” or “T cells” belong to a group of white blood cells known as lymphocytes, and play a central role in cell-mediated immunity. They can be distinguished from other lymphocyte types, such as B cells and natural killer cells (NK cells) by the presence of a special receptor on their cell surface called a T cell receptor (TCR), of which there are many, however molecular tools exist to identify the presence of differing TCRs by DNA sequence analysis of the genomic DNA from the lymphocytes.

A “cluster of differentiation” (often abbreviated as CD) is a protocol used for the identification and investigation of cell surface molecules present on white blood cells, providing targets for immunophenotyping of cells. Physiologically, CD molecules can act in numerous ways, often acting as receptors or ligands (the molecule that activates a receptor) important to the cell. A signal cascade is usually initiated, altering the behavior of the cell (see cell signaling). Some CD proteins do not play a role in cell signaling, but have other functions, such as cell adhesion. The number of CD proteins for humans is numbered up to 350 most recently (as of 2009).

“CD8” (cluster of differentiation 8) is a transmembrane glycoprotein that serves as a co-receptor for the T cell receptor (TCR). Like the TCR, CD8 binds to a major histocompatibility complex (MHC) molecule, but is specific for the class I MHC protein. There are two isoforms of the protein, alpha and beta, each encoded by a different gene. In humans, both genes are located on chromosome 2 at position 2p12.

“T cell receptor” or “TCR” is a molecule found on the surface of T lymphocytes (or T cells) that is, in general, responsible for recognizing antigens bound to major histocompatibility complex (MHC) molecules. The binding between TCR and antigen is of relatively low affinity and is degenerate: that is, many TCR recognize the same antigen and many antigens are recognized by the same TCR. The TCR is composed of two different protein chains (that is, it is a heterodimer). In 95% of T cells in peripheral blood, this consists of an alpha (α) and beta (β) chain, whereas in 5% of T cells in peripheral blood, this consists of gamma and delta (γ/δ) chains. These percentages are different in other locations in the body.

“VDJ recombination” is also known as somatic recombination, is a mechanism of genetic recombination in the early stages of antibody (also referred to as immunoglobulin (Ig)) and T cell receptor (TCR) production of the immune system. Antibodies and TCRs have structures in common that are designed to bind to antigens, which are components of any matter that are capable of inducing an immune reaction. Both antibodies and TCRs are formed, in part, by what is termed in the art as V(D)J recombination, which refers to the Variable, Diverse, and Joining gene segments found in vertebrates, of which there are a plurality of variant copies of each. The V(D)J gene segments are employed by an individual's immune system to create numerous different binding structures for portions of different antigens, generated and employed as and when the immune system is challenged by a new antigen. The great variety of such binding structures needed for the many, many different possible antigens is generated by a nearly-random mechanism that selects one variety of the V gene segment and one variety of the J gene segment, marries them to a selected variety of the D gene segment, thereby generated DNA that encodes diverse but related proteins to match antigens from bacteria, viruses, parasites, dysfunctional cells (such as tumor cells or the consequences of inflammatory processes), pollens, and many, many more. One particularly peculiar learning of molecular immunology over the past few decades is that we now know that antibody-forming cells (B cells) and TCR-carrying cells have an altered genome due to the just described mechanism for forming a plethora of different binding sites on antibodies and TCRs. The particular V(D)J combination is established by the encoding DNA and is fixed in its position in the individual's B cell and T cell genomes, which no longer are identical to the genomes of nearly all the other cells in that individual. (Until this antibody binding site phenomenon was elucidated in the late 1970s, biologists held as true that all cells of an individual shared the same DNA sequences that were placed together upon the fertilization of an egg by a sperm that formed the embryo that became the individual.) The difference that is fixed in the genome of the T cells allows us to measure the relative abundance of different T cell clonotypes by sequencing the VDJ region and measuring the concentrations thereof inter se.

The “Gleason Grading system” is used to help evaluate the prognosis of men with prostate cancer. Together with other parameters, it is incorporated into a strategy of prostate cancer staging which predicts prognosis and helps guide therapy. A Gleason score is given to prostate cancer based upon its microscopic appearance. Cancers with a higher Gleason score are more aggressive and have a worse prognosis.

“Robotic-mediated radiotherapeutic procedures” can be accomplished readily using a commercially available robotic system that is marketed under the brand name “CyberKnife” by Accuray Incorporated, Sunnyvale, Calif. The CyberKnife system is used for treating benign tumors, malignant tumors and other medical conditions. The two main elements of the CyberKnife are (1) the radiation produced from a small linear particle accelerator and (2) a robotic arm which allows the energy to be directed at any part of the body from any direction. The CyberKnife system is a method of delivering radiotherapy, with the intention of targeting treatment more accurately than standard radiotherapy.

“High-Intensity Focused Ultrasound” (HIFU) or “high frequency ultrasound” is a highly precise medical procedure using high-intensity focused ultrasound to heat and destroy pathogenic tissue rapidly by raising the tissue's immediate temperature in excess of 45° C. It is one modality of therapeutic ultrasound.

A “biopsy” is a medical test involving the removal of cells or tissues for examination. It is the medical removal of tissue from a living subject to determine the presence or extent of a disease. The tissue is generally examined under a microscope by a pathologist, and can also be analyzed chemically. When an entire lump or suspicious area is removed, the procedure is called an excisional biopsy. When only a sample of tissue is removed with preservation of the histological architecture of the tissue's cells, the procedure is called an incisional biopsy or core biopsy. When a sample of tissue or fluid is removed with a needle in such a way that cells are removed without preserving the histological architecture of the tissue cells, the procedure is called a needle aspiration biopsy.

A “radical prostatectomy” is the surgical removal of the prostate gland in order to remove prostate cancer.

“Radiation therapy”, “radiation oncology”, or “radiotherapy” sometimes abbreviated to XRT, is the medical use of ionizing radiation, generally as part of cancer treatment to control malignant cells. Radiation therapy is commonly applied to the cancerous tumor because of its ability to control cell growth. Ionizing radiation works by damaging the DNA of exposed tissue, furthermore, it is believed that cancerous cells may be more susceptible to death by this process as many have turned off their DNA repair machinery during the process of becoming cancerous. To spare normal tissues (such as skin or organs which radiation must pass through in order to treat the tumor), shaped radiation beams are aimed from several angles of exposure to intersect at the tumor, providing a much larger absorbed dose there than in the surrounding, healthy tissue. Besides the tumor itself, the radiation fields may also include the draining lymph nodes if they are clinically or radiologically involved with tumor, or if there is thought to be a risk of subclinical malignant spread. It is necessary to include a margin of normal tissue around the tumor to allow for uncertainties in daily set-up and internal tumor motion. These uncertainties can be caused by internal movement (for example, respiration and bladder filling) and movement of external skin marks relative to the tumor position.

In biology, a “clonotype” is a collection of samples that are substantially similar and/or identical (i.e. clonal).

“Formalin-fixed, paraffin-embedded” (“FFPE”) tissues are a common way to preserve tissue samples.

An “adjuvant” is a pharmacological or immunological agent that modifies the effect of other agents, such as a drug or vaccine. They are often included in vaccines to enhance the recipient's immune response to a supplied antigen, while keeping the injected foreign material to a minimum. Adjuvants in immunology are often used to modify or augment the effects of a vaccine by stimulating the immune system to respond to the vaccine more vigorously, and thus providing increased immunity to a particular disease. Adjuvants accomplish this task by mimicking specific sets of evolutionarily conserved molecules, so called PAMPs, which include liposomes, lipopolysaccharide (LPS), molecular cages for antigen, components of bacterial cell walls, and endocytosed nucleic acids such as double-stranded RNA (dsRNA), single-stranded DNA (ssDNA), and unmethylated CpG dinucleotide-containing DNA. Because immune systems have evolved to recognize these specific antigenic moieties, the presence of an adjuvant in conjunction with the vaccine can greatly increase the innate immune response to the antigen by augmenting the activities of dendritic cells (DCs), lymphocytes, and macrophages by mimicking a natural infection. Furthermore, because adjuvants are attenuated beyond any function of virulence, they pose little or no independent threat to a host organism.

Additional details, features, characteristics and advantages of the invention are disclosed in the following examples that, in an exemplary fashion, show embodiments of the present invention. However, these examples should by no means be understood as to limit the scope of the invention.

### Example 1

This example illustrates a protocol for collecting outcome information for different medical treatments of prostatic cancer patients, where each treatment includes use of autologous engineered T cells.

Patients are recruited under an institutional review board (IRB) approved protocol for human subjects. Written informed consent is obtained from each patient.

Patient eligibility criteria are as follows: histologically confirmed adenocarcinoma of the prostate. We obtain a clinically diverse set of patients in order to analyze the relationship between the appearance of induced clonotypes to the clinical characteristics of the patient. This diversity includes patients with localized and non-localized disease. This diversity includes patients with or without prior history of treatment (e.g., hormone deprivation treatment). This diversity includes a range of stages of the disease.

We obtain a complete medical history at the time of intervention (where possible), including PSA (Prostate Specific Antigen) measurements, tumor grading/scoring information such as Gleason scores, etc., per standard urological practice. Similarly, we obtain the follow-up clinical information after the procedure, including longitudinal PSA.

We analyze the relationship between the appearance of induced clonotypes and the course of disease and response to treatment following the intervention.

Medical procedures include the following: cryosurgery, radical prostatectomy, prostate biopsy, radiation therapy, brachytherapy, robotic-mediated radiotherapy using, for example, the CyberKnife Robotic Radiosurgery System (Accuray Incorporated, Sunnyvale, Calif.), electroporation, high frequency ultrasound (HIFU), photodynamic therapy, prostate laser surgery, androgen deprivation therapy, chemotherapy, and others. Note that prostate biopsies are typically considered to be diagnostic procedures rather than therapeutic procedures, but it is known that circulating tumor cells may increase in the days after a biopsy, so in this context we include it in our list of medical procedures of interest. Also, note that radical prostatectomies induce an immune response, even with the removal of tumor tissue, due to tumor tissue shedding caused by the surgery.

We obtain as complete a description of the procedure as possible. For cryosurgery, this includes the thermal timecourse used (which includes number of freezings, speed of freezings, etc.), the spatial extent and direction of the freezings, and other clinical parameters of the intervention, including adjuvants and/or medications. We obtain similar information for each intervention, in order to analyze the relationship between the clinical parameters of the intervention and the appearance of induced clonotypes.

We obtain a minimum of two blood samples—one prior to the medical procedure and one after the medical procedure at or about 7 days afterwards. Where possible, we obtain one or more additional samples at later time points. Where possible, we obtain more than one prior blood sample. These blood samples are peripheral blood, obtained by venipuncture. Blood from other locations is typically much less readily available. However, if blood from additional locations is available, at the discretion of the treating physician, we obtain additional blood samples.

These other locations may potentially include tumor infiltrating lymphocytes from the tumor tissue, blood cells from lymph nodes (e.g., if a lymph node biopsy is done), or from other organs from which blood cells are available.

Sample collection is as follows: each sample consists of 10 mL of blood. We isolate peripheral blood mononuclear cells by by a standard centrifugation technique for cell separation on Ficoll-Hypaque Density Media (Sigma-Aldrich, St. Louis, Mo.). We enrich multiple lymphocyte subsets from freshly isolated peripheral blood mononuclear cells by immunomagnetic selection with microbeads using a kit designed to isolate CD8+ T cells (Catalog #130-096-495 available from Miltenyi Biotec Inc., Auburn, Calif.) or other such kits designed for isolation of other lymphocytes; other separation methods can also be used, such as Fluorescence-Activated Cell Sorting (FACS), for which a number of companies provide reagents and instruments. See, e.g., Beckman Coulter, Inc. (Indianapolis, Ind.), Becton, Dickinson and Company (Franklin Lakes, N.J.), among other providers. We isolate one or more of the following subsets: B cells, CD8+ T cells, CD4+ T cells, CD4 Th1 cells, CD4 Th2 cells, CD4 Th17 cells, Treg cells (nTreg, iTreg, Th3, Trl), NKT cells, and/or gamma-delta T cells. Depending on blood volumes, we may separate into subsets based on naïve, effector, or memory subsets, as known in the art.

We extract total genomic DNA from sorted cells using standard protocols incorporated into the QIAamp DNA Blood Mini kit (QIAGEN Inc., Valencia, Calif.) or a similar kit, or a commercial service that provides DNA extraction or isolation (e.g., BioServe Biotechnologies, Ltd., Beltsville, Md., among many other such providers). Sequencing of the isolated DNA is accomplished by sending samples to a DNA sequencing service provider, such as Adaptive Biotechnologies Corporation (Seattle, Wash.), which specializes in sequencing and characterizing lymphocyte populations; other DNA sequencing service providers include SeqWright, Inc. (Houston, Tex.) or any of the many other GLP-compliant facilities available. We prepare and ship DNA for sequencing per vendor instructions: at a concentration of approximately 50 ng/μL, with an absorbance ratio (A_{260/280}) ratio of at least 1.8, and shipped on dry ice using a vendor-supplied shipping container.

Note that this procedure results in pooled genomic DNA. Alternative methods such as high throughput microdroplet-based analysis (RainDance Technologies, Inc., Lexington, Mass.) provide single cell sequencing rather than pooled sequencing. With pooled sequencing, we carry out an additional analysis step for pairing receptor chains, described in detail below. However, when single cell sequencing is commercially available, we extract genomic DNA using single cell technology per vendor instructions.

From sequence data of the complementarily-determining region 3 (CDR3) of the T cell receptor (TCR) provided by the sequencing vendor, we identify induced clonotypes, as follows. We define an induced clonotype as a clonotype whose change in frequency from a prior sample to a post-intervention sample is above the defined threshold of 0.5%. This threshold is a conservative frequency that is used to define a highly expanded clone; it is supported by vendor reproducibility data as shown in FIG. 4 (in this case, the vendor was Adaptive Biotechnologies Corporation, identified above). The analysis is also repeated with higher and lower thresholds (down to 0.1%). We rank and characterize clonotypes as weakly or strongly emergent based on their percentage increase in frequency. For example, a clonotype which had a frequency of 0.1% prior to intervention and 0.9% after intervention has an increase in frequency of 0.8%; since this is greater than the minimum increase of 0.5%, we characterize this clonotype as induced.

We construct an autologous engineered T or B cell using the induced clonotype information that we identify as described above. This engineered T or B cell is the basis of one immunotherapy further described herein, which we test in vitro and, with prior IRB approval, will use therapeutically in vivo.

With induced or expanded clonotypes, we further characterize the lymphocyte receptors by the following criteria: Several receptors consist of two chains, which are paired in vivo. For example, in T cells, a receptor may consist of an alpha and a beta chain; a different receptor may consist of a gamma and a delta chain. In B cells, the two chains are the heavy chain and the light chain. In the following explanation, for convenience, we refer to alpha and beta chains, but a similar strategy is used for pairing heavy chains and light chains, or gamma chains and delta chains.

We sequence these chains as described above. Note that if single cell or single clonotype sequencing is available, this pairing step is not necessary—if cells are sequenced individually, the chain pairing is known without further effort. If pooled sequencing is used, then we have a list of induced alpha chains, and a list of induced beta chains, (or heavy chains and light chains, etc.), and we now pair the chains.

In order to pair the chains, we benefit from the fact that these chains are made in vivo via VDJ recombination. Furthermore, V and J gene segment-specific antibodies are readily available from commercial sources, including Life Technologies Corporation (Grand Island, N.Y.). Therefore, we start with one chain—for example, the beta chain. We identify an induced clonotype, and from its sequence, we identify its V and J gene segments, as follows.

We annotate the induced clonotype's gene segments from its sequence using specialized, publicly available immunology gene alignment software, including the following: tools available from the International IMmunoGeneTics information system (IMGT; Lefranc, et al., NUCL. ACIDS RES. 37:D1006-D1012 (2009)), including V-Quest (Brochet, et al., NUCL. ACIDS RES. 36: W503-508 (2008)) and JunctionAnalysis (Yousfi Monod, et al., BIOINFORMATICS 20:1379-i385 (2004)); JOINSOLVER (Souto-Carneiro, et al., J. IMMUNOL. 172(11):6790-6802 (2004)); VDJSolver (Ohm-Laursen, et al., IMMUNOLOGY 119(2):265-77 (2006)); Somatic Diversification Analysis (SoDA; Volpe, et al., BIOINFORMATICS 22(4):438-44 (2006)); iHMMune-align (Gaeta, et al., BIOINFORMATICS 23:1580-1587 (2007)), and other similar tools.

We obtain antibodies for the identified segments. We use the antibodies to purify a subset of cells which express that gene segment in their (surface) receptors (e.g., using FACS, or immunomagnetic selection with microbeads). Finally, using this subset of cells which have been purified for the desired gene segments, we sequence again, as described above, or, alternatively, in a less expensive, more low-throughput manner. If necessary, we sequence this subset at a higher resolution than previously. In this new data set, fewer induced clonotypes are identified, which eases the task of chain pairing. Depending on the gene segments, there may be only one induced alpha chain and one induced beta chain for example.

Alternatively, (e.g., if the above method is inconclusive), we isolate our gene segment purified cells (or unpurified cells) in a microwell plate, where each microwell has a very low number of cells of between about 10 and 1. We amplify and sequence the cells in each well individually, which provides another means to pair the chains of interest by sequencing on a single cell basis. We alternatively amplify and sequence the cells in each well on a single clonotype basis if we treat the cells with suitable adjuvants to induce proliferation of the cells in the microwells prior to sequencing them.

Once we pair the alpha/beta, gamma/delta, or heavy/light chains of the induced clonotypes, we engineer an autologous B or T cell which expresses a receptor corresponding to the induced clonotype. This receptor may be a T cell receptor on a T cell surface, or a chimeric antibody receptor. The chimeric antibody receptor is comprised of a single chain variable fragment (scFv) on the T cell surface. The chimeric antibody is enhanced by the presence of co-stimulatory endodomains.

The following procedure describes the engineering of a T cell receptor on a T cell surface. The tools for this process are commercially available and a great deal of literature describes this process and is well known to those skilled in the art. In particular, recently published work with T cells and chimeric antigen receptors (CARs; engineered receptors) provides rich guidance regarding methods usefully employed in accordance with the present disclosure for enhancing the potency of the engineered T cells (in particular, using third generation CARs). See, e.g., Porter et al., “Chimeric Antigen Receptor-Modified T Cells in Chronic Lymphoid Leukemia,” N. ENGL. J. MED. 365(8):725-733 (2011).

When engineering a T cell which expresses a desired (induced) T cell receptor sequence, we prepare by acquiring a suitable lentiviral or other retroviral vector. Frecha et al., “Advances in the Field of Lentivector-based Transduction of T and B Lymphocytes for Gene Therapy,” MOLECULAR THER. 18(10):1748-1757 (2010). A number of commercial vendors provide customized lentiviral vectors, and a number of kits for lentiviral transduction are available, including Cell Biolabs, Inc. (San Diego, Calif.). In the alternative, other sorts of vectors can be usefully employed including, for example, a gamma retroviral vector in view of mounting data showing these vectors to be safe when expressed in human T cells, for example. See Powell and Levine, “Genetically Engineered Antigen Specificity in T Cells for Adoptive Immunotherapy,” in J. Medin and D. Fowler, Eds., Experimental and Applied Immunotherapy, Chapter 12 (Humana Press, 2011). Another alternative for engineering the autologous T cells is to introduce the foreign DNA specific to the TCR of interest using electroporation-mediated DNA or mRNA transfection, which are methodologies that are well-known to the skilled artisan. Recent reports have indicated, for example, greater than 90% transduction efficiency introducing tumor antigen-specific TCR genes that conferred tumor reactivity to previously nonreactive, unstimulated human T cells. Id.

Our general approach in creating genetically engineered autologous T cells that express TCR or suitable CAR sequences is to have commercially-engineered lentiviral particles created in which the desired (induced) TCR or CAR sequences have been introduced. In addition, we acquire a suitable population of T cells from the patient via leukapheresis, and maintain them ex vivo.

Following vendor instructions, we then incubate the T cell population with the lentivirus. Commonly, cytokines such as IL-2 or IL-7 are used to facilitate this process; in this case, we follow vendor instructions.

We confirm the success of the transduction, and the expression of the engineered T cells in multiple ways. First, we use VDJ gene segment specific antibodies, as described previously. Second, we sequence the engineered cells, as described previously. We use additional verification methods as appropriate.

We test our engineered T cells in vitro by incubating them with tumor tissue lysate and also with various combinations of adjuvants such as GM-CSF, IL-2, and others. We measure efficacy using assays for cytokines (e.g., IFN-gamma) and T cell proliferation. These assays are commercially available, and we follow vendor instructions.

Finally, for our in vivo study, we follow FDA guidance and Good Manufacturing Procedures to produce engineered T cells for in vivo use. We adhere to extensive regulatory guidance in developing the necessary procedures. In vivo efficacy is measured through surrogate endpoints (e.g., longitudinal PSA, circulating tumor cells) and clinical endpoints (e.g., overall survival, progression free survival, tumor regression).

### Example 2

This example illustrates the efficacy of a cryosurgical and immunotherapeutic procedure based on a rodent study.

Cryosurgery and immunotherapy as well as a combination thereof was performed on a rat model for human prostate cancer, using rat prostatic tumor tissue (Dunning R3327). The Dunning R3327 rat carcinoma is a well-recognized model for human prostate adenocarcinoma. Sinowatz et al., PROSTATE 19(4):273-278 (1991).

The rats used for the procedure were Male Copenhagen rats between 8 and 15 weeks of age. The rats and rat food were purchased from Harlan Teklad (Madison, Wis.). The Dunning Cell line was obtained from Dr. Israel Barken (San Diego, Calif.) and propagated in RPMI 1640 media (Sigma-Aldrich, St. Louis, Mo.) with 10% Fetal

Bovine Serum (FBS; Sigma-Aldrich, St. Louis, Mo.) in 5% CO₂ in air at 37° C. Tumor cells were subcutaneously implanted in rats with 2×10⁵ cells on the left side of the rat (about ½ inch from the inguinal area) and 2×10⁴ cells on the right side. When tumors on the right side reached about 5 mm³ to 7 mm³ in size the rats were divided into four groups. Table 1 depicts the number and purpose of each group.

TABLE 1
Study Design

| Groups | No. of rats | Immunotherapy | Cryosurgery |
|---|---|---|---|
| Control | 7 | None | No |
| Crosurgery Only | 7 | None | Yes |
| Cryosurgery + IS | 7 | IL-2 5000 IU + | Yes |
| | | GM-CSF 1000 IU | |
| IS, only | 7 | IL-2 5000 IU + | No |
| | | GM-CSF 1000 IU | |

Groups 2 and 3 received cryosurgery under anesthesia. The anesthesia for cryosurgery injected intramuscularly was a mixture of Acepromazine, Xylazine, and Ketamine mixed at a ratio of 0.24 to 0.76 to 1.0, respectively, at a dose of 1 mL/kg. The cryosurgical system used was the CRYOcare CRYOsurgical System catalog number 770-101 (Boston Scientific Corporation, Watertown, Mass.). A cryosurgical model of 25% freezing was chosen. Table 2 depicts the cryosurgical conditions used on the rats.

TABLE 2
Cryosurgical Conditions for Individual Animals in Treated Groups

| | | Probe | Seconds of | Temperature |
|---|---|---|---|---|
| Group | Animal | (−C. °) | freeze (″) | (C. °) |
| Cryosurgery | 1 | 122 | 50″ | 16.3 |
| alone | 2 | 118 | 50″ | 32.6 |
| | 3 | 118 | 50″ | 59.7 |
| | 4 | 120 | 50″ | 60.1 |
| | 5 | 119 | 50″ | 48.7 |
| | 6 | 127 | 50″ | 35.2 |
| | 7 | 120 | 50″ | 35.4 |
| Cryosurgery + | 1 | 128 | 50″ | 52.4 |
| Immunotherapy | 2 | 118 | 50″ | 40.1 |
| | 3 | 116 | 50″ | 35.6 |
| | 4 | 124 | 50″ | 62.1 |
| | 5 | 127 | 50″ | 58.2 |
| | 6 | 127 | 50″ | 53.6 |
| | 7 | 117 | 50″ | 42.3 |

Approximately one day following cryosurgery, each rat from groups 3 and 4 were injected subcutaneously with 5000 injection units (IU) of interleukin-2 (IL-2) and 1000 IU of granular macrophage colony stimulating factor (GM-CSF) approximately ¼ inch from the tumor sit towards the inguinal node on the right side. The rats received injections at these concentrations of IL-2 and GM-CSF for 14 consecutive days.

Tumor dimensions were measured 3 times a week for 60 days by measuring the perpendicular minor dimension (W) and major dimension (L) using sliding calipers. Approximate tumor volume was calculated by the formula W²×L×½. A one-way analysis of variance (ANOVA) test with an α=0.05 was used to evaluate efficacy of the treatment. Animal survival was analyzed using the Log-rank analysis with an α=0.05. Metastasis and tumor cure was analyzed using a two-way Fisher exact test with an α=0.05.

Table 3 illustrates that cryosurgery was more effective against the Dunning 3327 prostate cancer when combined with the immunotherapeutics IL-2 and GM-CSF.

TABLE 3
Comparison of the mean tumor volumes on the left
side between the treated groups and control

| | | Mean tumor | | Mean tumor | |
|---|---|---|---|---|---|
| | | volume | | volume | |
| | No. of | (mm³) | *p- | (mm³) | *p- |
| Groups | rats | on Day 0 | value | on Day 21 | value |
| Control | 6 | 430 ± 186 | — | 43478 ± 14570 | — |
| Cryosurgery | 6 | 453 ± 154 | 0.492 | 4212 ± 10317 | 0.000 |
| Cryosurgery + | 6 | 336 ± 101 | 0.283 | 0 ± 0 | 0.000 |
| Immunotherapy | | | | | |
| Immunotherapy | 6 | 402 ± 65 | 0.488 | 57499 ± 12736 | 0.065 |

*Control vs the treated groups

Table 4 shows a tumor cure rate in the rats receiving the combined treatment reached 80%.

TABLE 4
Comparison of Tumor Cure Rates

| Groups | | No. of rats | Tumor cure rate (%) | *p-value |
|---|---|---|---|---|
| 1 | Control | 6 | 0% (0/6) | — |
| 2 | Crosurgery Only | 6 | 50% (3/6) | 0.182 |
| 3 | Cryosurgery + IS | 5 | 80% (4/5) | 0.015 |
| 4 | IS, only | 5 | 0% (0/5) | 1.000 |

*Control vs the treated groups

Table 5 shows that the rats receiving the combined treatment had a significant increase in survival time as compared to the rats in other groups.

TABLE 5
Comparison of Mean Survival Times
for Control and Treated Groups

| | | | | Log-rank |
|---|---|---|---|---|
| | | | Mean animal | analysis |
| Groups | | No. of rats | survival time (days) | (*p-value) |
| 1 | Control | 6 | 34.7 ± 12.4 | — |
| 2 | Crosurgery Only | 6 | 100.5 ± 71.9 | 0.068 |
| 3 | Cryosurgery + IS | 5 | 140.4 ± 57.2 | 0.010 |
| 4 | IS, only | 5 | 30.6 ± 2.7 | 0.705 |

*Control vs the treated groups

Without being limited to any particular theory, the inhibition of cryosurgery on the Dunning 3327 tumors was by enhancing immune function of the rats in combination with immune stimulating agents IL-2 and GM-CSF.

This model may prove to be effective for human prostate adenocarcinomas. Furthermore, the medical procedures performed is not to be restricted to cryosurgical procedures and may include other procedures such as radical prostatectomy, prostate biopsy, radiation therapy, brachytherapy, robotic-mediated radiosurgery using, for example, the CyberKnife Robotic Radiosurgery System (Accuray Incorporated, Sunnyvale, Calif.), electroporation, high frequency ultrasound (HIFU), photodynamic therapy, prostate laser surgery, androgen deprivation therapy, chemotherapy, and others. The range of cytokines used is also not to be understood as limited to IL-2 and GM-CSF and may include IL-4, IL-6, IFN-γ, and others.

### Example 3

This example illustrates a protocol for identifying lymphocyte clonotypes that are usefully employed in one embodiment of the present invention.

Heparinized venous blood is layered onto a Ficoll-Isopaque (Lympho separation Medium, ICN Bio medicals, Ohio) in a 15 mL conical tube in the ration of 3:1. The tubes are centrifuged at 1800 rpm for 20 minutes and the middle layer is removed and transferred to another tube. The middle layer is then centrifuged at 2000 rpm for 10 minutes. The supernatant is discarded and the pellet is suspended in RPMI-1640 provided by ICN Bio chemicals, Ohio. The cells are washed twice with RPMI-1640 and suspended in 1 mL of RPMI-1640. The cell suspensions are divided into two tubes and incubated with fluorescein isothiocyanate (FITC) or phycoerythrin (PE) labeled anti-CD4 or anti-CD8 monoclonal antibodies manufactured by Pharmingen, San Jose, Calif. The tubes are mildly vortexed and incubated for 2 hours in the dark at room temperature. CD4⁺ and CD8⁺ cells are identified based on staining with specific antibodies and sorted on Fluorescence-Activated Cell Sorting (FACS) Star Plus manufactured by Becton Dickinson, San Jose, Calif. The sorted CD4⁺ and CD8⁺ cells are then washed with phosphate buffered saline (PBS).

### Example 4

This example illustrates basis for identifying B cell or T cell mixtures that include those having receptors that have increased in abundance using non-sequencing methods.

The general strategy of the present invention is to study the immune system's response to an event that triggers an immune response, necessarily starting with what is referred to herein as an immune recognition event. Immune recognition events can be induced by injury or a medical procedure or intervention or other event. Studying the result of the immune recognition event provides the information needed to design and produce a tailored therapeutic intervention that, in one embodiment, induces a stronger and more directed immune response to the antigens that the immune system itself organized itself to attack.

One embodiment of the present invention (described above) is to sequence the T and/or B cell receptor repertoire and then genetically engineer a T cell- and/or B cell-based therapy for infusion into the patient whose receptor repertoire was studied. Another embodiment of this approach replaces T cell receptor or B cell receptor sequencing with spectratyping (also known as immunoscoping).

Spectratyping analysis is a method for quantifying the presence of lymphocytes with particular V and/or J segments, as set forth in Kepler et al., “Statistical Analysis of Antigen Receptor Spectratype Data,” BIOINFORMATICS 21(16):3394-3400 (2005) at FIG. 1 on p. 3395.

There are multiple ways to carry out spectratyping analysis, including quantitative PCR, flow cytometry with V and/or J segment specific antibodies, and the like. It is important to note that some forms of spectratyping reveal not just the quantity of cells with a given V and J segment, but also the oligoclonality within each particular combination of V and/or J segments. The quantification of V and/or J segments can also be done using sequencing—starting with the sequence reads, the V and J segments can be calculated in silico (e.g. using IMGT tools such as V-Quest and IMGT HighV-Quest, which were described and referenced above).

One advantage of spectratyping is that it is faster than sequencing. One advantage of sequencing is that it reveals the diversity (i.e., the oligoclonality) of an individual V-J “compartment.” In other words, a given subpopulation of lymphocytes expressing a particular V and/or a particular J segment may be composed of mostly one expanded clone or a large number of different cells from different clones. Sequencing data clarifies the content of the V-J compartment—but a perfectly valid and useful immunotherapeutic cell-based reagent can be identified and used without necessarily having the sequence data.

Another embodiment of the present invention replaces the genetic engineering step with a filtering/purification step, in which the patient's own lymphocytes are filtered to isolate lymphocytes bearing a particular V and/or J segment. This filtering can be done readily using a variety of technologies well-known in the art, including FACS, magnetic beads, and others. These techniques are enabled by commercially-available antibodies to various human V and J segments from, for example, Pierce Antibodies from Thermo Fisher Scientific (Rockford, Ill.).

As noted, a first objective is to identify those T cells or B cells, or both, that increase in abundance in a patient's blood after an immune recognition event, such as a medical procedure, intervention or event; and then identify a particular combination of V and/or J segments in the T cell or B cell repertoires that has expanded and/or become more oligoclonal. A subset of cells expressing the identified V and/or J segments is then isolated from the patient's own blood, expanded, stimulated, or modified ex vivo, and then reinfused as a therapeutic treatment.

Identification of lymphocytes having the particular combination of V and/or J segments that expands post-immune recognition event is accomplished by spectratyping the patient's lymphocytes before and after immune recognition event. In so doing, a particular combination of V and/or J segments in the T cell or B cell repertoires which has expanded and/or become more oligoclonal is identified. A subset of cells expressing the identified V and/or J segments is then isolated from the patient's own blood, expanded, stimulated, and then reinfused as a therapeutic treatment.

The protocol using spectratyping to identify one or more lymphocyte groupings that expanded in abundance after the immune recognition response has an optional step for modifying the autologous lymphocyte ex vivo. Modifications usefully employed in the context of the present invention include engineering the autologous lymphocytes to tweak the TCR or BCR activity by adding a CAR, for example.

A key advantage of this protocol using spectratyping to identify the cell-based immune-reagent composed of a particular V and/or J subset of lymphocytes, is that the isoation step can be done very quickly (relativeto sequence based approaches requiring engineering of the autologous lymphocytes—not as an option but a necessary step).

Another variation on this theme is to identify an expanded receptor using before and after immune recognition event sequencing of DNA from the sets of lymphocytes, then carry out a “panning experiment” in which many copies of the identified receptor are incubated with a combinatorial library of possible epitopes. This approach relies on methods well-known in the art, including Sung et al., “T-Cell Epitope Prediction with Combinatorial Peptide Libraries,” J. COMPUT. BIOL. 9(3):527-39 (2002); and Pira et al., “High Throughput T Epitope Mapping and Vaccine Development,” J. BIOMED. BIOTECHNOL. 2010:325720 (2010). Once the corresponding epitope is identified, tetramers loaded with the identified epitope is used to isolate lymphocytes from the patient's own cells which are specific to that epitope (which should be mainly composed of the expanded receptor identified previously). These cells are then expanded ex vivo, manipulated (e.g. with cytokines), and then therapeutically infused into the patient.

The advantage of this approach is that it avoids the difficulties associated with genetic engineering (at the cost of having to identify the epitope associated with the lymphocyte receptor that sequencing reveals to be expanded.)

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents are covered thereby.

What is claimed is:

1. A method of treatment of a patient who suffers from a disease or injury, comprising the steps of:
(a) recording or carrying out an immune-recognition event in the patient; and
(b) administering an immunostimulating reagent substantially proximate to the time of the immune-recognition event.

2. The method of claim 1, wherein the immunostimulating reagent is one or more cytokines.

3. The method of claim 1, further comprising the steps of:
(a) drawing a first blood sample from a patient prior to the immune-recognition event;
(b) drawing a second blood sample from the patient after the immune-recognition event; and
(c) identifying lymphocyte clonotypes that have expanded following the immune-recognition event;
wherein the immunostimulating reagent comprises autologous lymphocytes relative to the patient.

4. The method of claim 3, further comprising the steps of:
(a) purifying lymphocytes from the first blood sample; and
(b) purifying lymphocytes from the second blood sample.

5. The method of claim 3, wherein the autologous lymphocytes include T cells with T cell receptors that are substantially identical to those of the expanded lymphocyte clonotypes.

6. The method of claim 5, wherein the autologous lymphocytes are genetically engineered to include T cell receptors that are substantially identical to those of the expanded lymphocyte clonotypes.

7. The method of claim 3, wherein the administering step is accomplished by intravenous infusion into the patient.

8. The method of claim 3, wherein the immune-recognition event is a medical or veterinary procedure, which immune-recognition event occurs after the drawing of the first blood sample and prior to the drawing of the second blood sample.

9. The method of claim 4, further comprising the steps of:
(a) isolating DNA samples from the purified lymphocytes of the first blood sample and the second blood sample;
(b) sequencing the respective DNA samples; and
(c) identifying lymphocyte clonotypes from the second blood sample that have expanded following the immune-recognition event.

10. The method of claim 9, wherein the immune-recognition event is induced by a surgical procedure selected from the group consisting of cryosurgery, radical removal of diseased or injured tissue, biopsy, robotic-mediated radiotherapeutic procedures, electroporation, bone marrow aspiration, and laser surgery.

11. The method of claim 9, wherein the immune-recognition event is induced by a nonsurgical procedure selected from the group consisting of radiation therapy, brachytherapy, high frequency ultrasound, and exposure to a drug employed in the context of photodynamic therapy, androgen deprivation therapy, or chemotherapy.

12. The method of claim 9, wherein the immunostimulating reagent comprises a culture of lymphocytes of the expanded lymphocyte clonotypes identified among the lymphocytes from the second blood sample.

13. A method for identifying lymphocyte clonotypes that have expanded following an immune-recognition event, comprising the steps of:
(a) drawing a first blood sample from a patient, wherein the first blood sample is drawn prior to the immune-recognition event;
(b) recording or carrying out the immune-recognition event on the patient;
(c) drawing a second blood sample from the patient, wherein the second blood sample is drawn subsequent to the immune-recognition event;
(d) spectratyping the purified lymphocytes; and
(e) identifying lymphocyte clonotypes that have expanded following the immune-recognition event.

14. The method of claim 14, further comprising the steps of:
(a) purifying lymphocytes from the first blood sample; and
(b) purifying lymphocytes from the second blood sample.

15. A method for treatment of a patient afflicted with a cancer, comprising the steps of:
(a) drawing a first blood sample from the patient, wherein the first blood sample is drawn prior to an immune-recognition event;
(b) carrying out a medical procedure on the patient, wherein the medical procedure induces the immune-recognition event;
(c) drawing a second blood sample from the patient, wherein the second blood sample is drawn subsequent to the medical procedure;
(d) identifying lymphocyte clonotypes that expanded following the medical procedure; and
(e) infusing lymphocytes characterized as being of one or more of the expanded lymphocytic clonotypes into the patient.

16. The method of claim 15, further comprising the steps of:
(a) purifying lymphoctyes from the first blood sample; and
(b) purifying lymphocytes from the second blood sample.

17. The method of claim 15, further comprising the step of selecting clonotypes with an expansion frequency of 0.5% or greater.

18. The method of claim 15, wherein the medical procedure is a surgical procedure selected from the group consisting of cryosurgery, radical removal of diseased or injured tissue, biopsy, robotic-mediated radiotherapeutic procedures, electroporation, and laser surgery.

19. The method of claim 15, wherein the medical procedure is a nonsurgical procedure selected from the group consisting of radiation therapy, brachytherapy, high frequency ultrasound, and exposure to a drug employed in the context of photodynamic therapy, androgen deprivation therapy, or chemotherapy.

20. The method of claim 1, further comprising the steps of:
(a) drawing a first blood sample from a patient prior to the immune recognition event;
(b) drawing a second blood sample from the patient after the immune-recognition event;
(c) identifying a set of one or more V and/or J segments whose usage has expanded between the “before” and “after” samples;
(d) isolating lymphocytes bearing the identified V and/or J segments;
(e) enhancing the efficacy of the isolated lymphocytes in vitro; and
(f) reinfusing the isolated and enhanced lymphocytes via reinfusion.
